(19) United States
(12) Patent Application Publication
(10) Pub. No.: US 2019/0307892 A1
(43) Pub. Date: Oct. 10, 2019

(54) TARGETED DRUG DELIVERY AND THERAPEUTIC METHODS USING APO-E MODIFIED LIPID NANOPARTICLES

(71) Applicant: Eriochem USA, LLC, Carson City, NV (US)

(72) Inventors: Jose Lucio Nunez, Parana (AR); Dante Selenscig, Parana (AR); Maria de los Angeles Ramirez, Parana (AR)

(73) Assignee: Eriochem USA, LLC, Carson City, NV (US)

(21) Appl. No.: 15/945,630

(22) Filed: Apr. 4, 2018

(51) Int. Cl. A61K 47/69; A61K 38/17; A61P 3/00; A61P 35/00; A61K 49/10; A61K 31/351; A61K 31/203; A61K 31/047; A61P 17/00

(52) U.S. Cl. CPC A61K 47/6909; A61K 38/1709; A61P 3/00; A61P 35/00; B82Y 5/00; A61K 31/351; A61K 31/203; A61K 31/047; A61P 17/00; A61K 49/108

(57) ABSTRACT

Methods for targeted delivery of therapeutic agents to a target cell or tissue with lipid nanoparticles comprising ApoE3. In embodiments, the invention specifically relates to targeted delivery of anticancer drugs, antibiotics, antifungal drugs, and diagnostic contrast agents, and associated treatment and diagnostic methods. In embodiments, diseases/conditions treated include those associated with over-expression of LDL receptors.

### INCORPORATION BY REFEENCE

Incorporated by reference herein is the entire disclosure and drawings of prior U.S. patent application Ser. No. 15/760,170, filed on Mar. 14, 2018, which is the National Phase of International Application No. PCT/US17/54045, filed on Sep. 28, 2017, which claims benefit of U.S. Provisional Application No. 62/402,632, filed on Sep. 30, 2016.

### FIELD OF THE INVENTION

The invention relates to targeted delivery methods for delivering therapeutic agents to target cells and tissues across the blood-brain barrier using lipid nanoparticles with apolipoproteins via LDL receptors. Methods of the invention include therapeutic treatment methods and diagnostic methods of diseases, particularly of the brain, and associated infections and conditions thereof.

### BACKGROUND

Targeted therapies are treatments that target specifics cells, without harming other cells in the body. These therapies represent major improvements in the clinical treatment of many diseases, including cancer, brain diseases, and various infections. Targeted therapies can lead to reduction of side effects (toxic effects) and reduction of dosage of administered drug, which results in less toxicity and costs. For example, many existing chemotherapeutic drugs, repurposed drugs and newly developed small molecule anticancer compounds which have high lipophilicity and low water-solubility are generally solubilized using high concentrations of surfactants and co-solvents, which frequently lead to adverse side effects.

Nanoemulsions are kinetically stable and suitable for parenteral delivery of poorly water-soluble anticancer drugs. In comparison to other nanocarriers, nanoemulsions are easier to prepare and do not necessarily require organic solvent/co-solvents; so the risk of carrier toxicity is low. However, nanoemulsions are manufactured using high energy procedures, such as sonication or high pressure homogenization and the nanoformulations often include multiple components to achieve several functions. Their scale-up production thus becomes significantly more costly and technically difficult since most commonly used laboratory techniques (such as sonication) are difficult to implement on a production scale. It is also quite challenging to obtain nanoparticles with a uniform size in a larger batch. (See Narvekar M. et at., AAPS Pharm. SciTech., Vol. 15, pp. 4822-4833 (2014)).

Prior methods for delivering drugs generally include: (a) liposome-based methods, wherein the therapeutic agent is encapsulated within the carrier; (b) synthetic polymer-based methods for creating particles having precise size characteristics; and (c) direct conjugation of a carrier to a drug, wherein the therapeutic agent is covalently bound to a carrier (such as, e.g., insulin).

Liposomes are small particles that form spontaneously when phospholipids are sonicated in aqueous solution, and consist of a symmetrical lipid bilayer configured as a hollow sphere surrounding an aqueous environment. Liposomes have a large carrying capacity, but are generally too large to effectively cross the blood-brain barrier (BBB), for example. Furthermore, liposomes are inherently unstable, and their constituent lipids are gradually lost by absorption by lipid-binding proteins in the plasma. Accordingly, attempts have been made to direct liposomes to particular cellular targets. As an example, immunoliposomes have been constructed in a process that involves covalent attachment of monoclonal antibodies (mAbs) to the surface of the liposome. Earlier studies have shown that the efficacy of liposome drug delivery appears to be inversely related to the diameter of the liposome particle. That is, the average HDL particle has a diameter of 10-20 nm. Hence, even the smallest liposomes have a diameter five times larger than the average HDL particle.

Lipoproteins are naturally occurring complex particles with a central core containing cholesterol esters and triglyceride surrounded by free cholesterol, phospholipids and apoproteins. These plasma lipoproteins can be divided into different classes based on size, lipid composition and apolipoproteins: chylomicrons, VLDL, IDL, LDL, HDL.

McChesney et al. (U.S. Patent Application Publication No. 2015/0079189) describe synthetic LDL nanoparticles comprising mixtures of phospholipids, triglycerides, cholesterol esters, free cholesterol and natural antioxidants, for selective delivering of lipophilic drugs to cellular targets expressing LDL receptors after intravenous injection for cancer treatment. These synthetic low density lipoprotein nanoparticles are also described as a lipid emulsion with a shelf life at 25° C. if greater than 1 year, and oral suspensions of about 2 years when stored in a sealed container and away from light exposure. These nanoparticles are prepared without any protein in order to avoid trigger clearance processes in the tissues of the reticuloendothelial system. Furthermore, these particles have a special coating layer that allows the particles to take the native lipoproteins as a coating; and after this coating the particles would be preferentially taken up by the targeted tissues.

Müller et al. (U.S. Pat. No. 6,288,040) describe the use of synthetic poly(butyl cyanoacrylate) particles to which ApoE molecules are covalently bound. The particle surface becomes further modified by surfactants or covalent attachment of hydrophilic polymers. Since these particles are not naturally occurring, they may have a variety of undesirable side effects. Furthermore, poly(butyl cyanoacyilate) is not an excipient approved by the FDA; and these particles use toxic surfactants such as Polysorbate 80 to cover the particle. Moreover, the described particles have a normal size of 300 nm. The presence of particles of about 300 nm of a synthetic material would likely trigger immune system responses.

Nelson et al. (U.S. Pat. No. 7,682,627) describe an artificial LDL for targeted carrier system for delivery across the blood-brain barrier. Specifically, Nelson describes a particle that has similar composition, size and behavior of an LDL, a method for manufacturing these particles and a method for producing conjugates of therapeutic agents with an LDL component to facilitate incorporation into LDL particle for transport across the BBB and subsequent release of the therapeutic agent into the cell. Conjugates include attachment of the therapeutic agent via an ester linkage that can be easily cleaved in the cytosol and consequently escape the harsh lysosomal conditions. These LDL particles comprised three elements: phospatidyl choline, fatty-acyl-cholesterol esters, and at least one apolipoprotein.

There are teachings indicating that individuals have different levels of Apo proteins in the body, and that these levels could also be affected by their physiological conditions. Thus, the amount of ApoE available to be adsorbed in these nanoparticles would be different in each individual (Liu H et al., 2015; Fidel Vila Rodriguez et al., 2011). The proportion of nanoparticles that would take the ApoE from the bloodstream and eventually reach the targeted tissue will also depend on the physiological characteristics of each individual and their condition.

In the field of targeted therapies, the nervous system—and the brain in particular—pose even more challenges. Due to a combination of protective effects of its body structures (skull and vertebral column), the meninges, and the blood-brain barrier, the central nervous system is extremely resistant to infection by bacterial pathogens. However, once an infection has initiated, the central nervous system is generally more susceptible than most other tissues, and host defense mechanisms that are normally seen in other areas of the body are inadequate in the central nervous system for preventing bacterial replication and progression of the disease process. Despite advances in diagnostic techniques and therapeutic methods, the combination of the bacterial virulence and a patient's immunostatus contributes to the high morbidity and mortality rates associated with bacterial infections affecting the central nervous system, and especially the brain.

The blood-brain barrier is a system-wide membrane barrier that prevents the brain uptake of circulating drugs, protein therapeutics, RnAi drugs, and gene medicines. Drugs can be delivered to the human brain for treatment of certain disease either by: (a) injecting the drug directly into the brain, thus bypassing the blood-brain barrier; or (b) injecting the drug into the bloodstream so that the drug enters the brain via the transvascular route across the blood-brain barrier. With intra-cerebral administration of the drug, it is necessary to perform a craniotomy, which requires drilling a hole in the head of the subject. In addition to being expensive and highly invasive, craniotomy-based drug delivery to the brain is also largely ineffective because the drug is only delivered to a tiny volume of the brain at the tip of the injection needle. The only way that a drug can be distributed widely in the brain is by the transvascular route following injection into the bloodstream. However, this approach requires the ability to undergo transport across the blood-brain barrier, which has proven to be a very difficult feat.

The transvascular approach for drug delivery remains the most ideal and noninvasive means to treat neurological diseases. Additionally, the most promising transvascular approach for drug delivery to the brain is by transporter molecules that deliver specific molecules without disrupting the blood-brain barrier.

The LDL receptors that bind ApoE have been found to be involved in transcytosis of LDL across the BBB. (Dehouck et al., 1997). ApoE-enriched liposomes have also been used to deliver Daunorubicin to cancer cells in mice based on the finding that tumor cells express high levels of LDL receptors on their membranes. (Versluis et al., 1999). Although Versluis et al. examined the tissue distribution of Daunorubicin, no data was presented relating to brain uptake, suggesting that transport of Daunorubicin across the blood-brain barrier was not envisaged.

Attempts have also been made to reduce toxicity of liposome formulations and to increase accumulation at the target site. In addition to liposome formulations of anti-tumor drugs, antifungal agents have also been targeted and commercialized (Abdus Samad, Y. Sultana and M Aqil, 2007). For example, use of Amphotericin B, a polyene antibiotic for treatment of systemic fungal infections, is associated with extensive renal toxicity. Amphotericin B acts by a mechanism in which it binds to sterol in the membrane of sensitive fungi, thus increasing the membrane permeability. The toxicity of this compound is due to non-specificity and binding to the mammalian cell cholesterol.

Recently, the first commercial preparation of Amphotericin B (AMBISOME) in the form of a liposome passed all clinical trials and is now conventionally used for the treatment of fungal infections. The liposomal Amphotericin B, by passively targeting the liver and spleen, reduces the renal and general toxicity encountered at normal dosage. However, renal toxicity appears when the drug is given at elevated dosages due to the saturation of liver and spleen macrophages. As another obstacle, many therapeutic agents suitable for treatment of diseases and disorders of the brain are frequently too hydrophilic to permit direct transport across the blood-brain barrier, and/or are susceptible to degradation in the blood and peripheral tissues.

Similar drawbacks are also prevalent in contrast agents. Despite their current value in providing main diagnostic information, current drawbacks include short blood half-life, nonspecific biodistribution, fast clearance, and slight renal toxicity. Although nanoparticles represent a promising strategy for non-invasive diagnosis, there remain concerns about their use in clinical procures due to potential issues of biological interactions, clearance routes, and coating of nanoparticles.

Therefore, there remains a need for targeted delivery of suitable therapeutic agents, particularly in the treatment of brain tissue disorders and diseases, and across the blood brain barrier. A similar need remains for improved methods of delivering contrast agents to target cells and tissues in diagnostic procedures. What is needed is an effective method of delivering therapeutic agents across the blood-brain barrier to target cells and tissues of the brain in order to deliver adequate amounts of drug(s) in a controlled manner, and preferably one that can be utilized in therapeutic as well as in diagnostic methods.

### SUMMARY OF THE INVENTION

It is an object of this invention to overcome the challenges encountered during delivery of certain therapeutic agents (drugs). Accordingly, described herein are methods for targeted delivery of active agents to the target tissue with lipid nanoparticles comprising ApoE3. In embodiments, the invention specifically relates to targeted delivery of anticancer drugs, antibiotics, antifungal drugs, and diagnostic contrast agents, and associated treatment and diagnostic methods. In embodiments, diseases/conditions treated include those associated with over-expression of r-LDL receptors.

In applications of the invention, ultrasonic contrast systems or agents may be used to detect physiological and pathological events by sensing the accumulation of the contrast agent at specific or targeted binding sites. In combination with the diagnostic applications, the present invention may additionally be applied for therapeutic purposes by delivering chugs to desired sites due to the specificity of the delivery system with the ability to further monitor the progress of the therapeutic treatment through repeated imaging at such target sites.

Specifically, the invention relates to methods for enhancing transport of a therapeutic agent to a target cell or tissue, comprising administering to a subject a lipid nanoparticle loaded with the therapeutic agent, the lipid nanoparticle comprising: a lipid core comprised of a triglyceride component and a cholesterol ester component; the therapeutic agent; a phospholipid layer; a surfactant coating layer surrounding the phospholipid layer and the lipid core; and a human recombinant apolipoprotein (ApoE3) adsorbed to a surface of the nanoparticle without Polysorbate 80, wherein: the lipid nanoparticle has preferential uptake in brain, lung, kidney and liver tissues that overexpress LDL receptors.

In some embodiments, a molar ratio of the therapeutic agent molecules per each recombinant ApoE3 molecule in the lipid nanoparticle is in a range of from 45-140. In some embodiments, the therapeutic agent is loaded in the lipid nanoparticle without conjugation. The target cell or tissue may be a cell or tissue that over-expresses LDL receptors; and the therapeutic agent may be a diagnostic magnetic resonance imaging contrast agent that accumulates at the target tissue due to the over-expression of LDL receptors.

The invention further relates to methods for enhancing transport of a therapeutic agent across a blood-brain barrier to a target cell or tissue, comprising administering to a subject a lipid nanoparticle loaded with the therapeutic agent, the lipid nanoparticle comprising: a lipid core comprised of a triglyceride component and a cholesterol ester component; the therapeutic agent; a phospholipid layer; a surfactant coating layer surrounding the phospholipid layer and the lipid core; and human recombinant apolipoprotein (ApoE3) adsorbed to a surface of the nanoparticle without Polysorbate 80, wherein: the therapeutic agent is transported to the target cell or tissue in a concentration that is at least 10 times greater than a concentration transported by the same lipid nanoparticle without human recombinant ApoE3 adsorbed thereto.

In embodiments, the target cell or tissue is a cell or tissue of the brain, and the therapeutic agent is a drug that does not reach the target cell or tissue in a therapeutic window when administered without the lipid nanoparticle. In other embodiments, the therapeutic agent is at least one diagnostic magnetic resonance imaging contrast agent that accumulates at the target brain tissue, and the method further comprises obtaining at least one magnetic resonance image of the target brain tissue. Where the therapeutic agent is a diagnostic contrast agent, the therapeutic agent may be a Gadolinium-based magnetic resonance imaging contrast agent or a magnetite-based magnetic resonance imaging agent coated with oleic acid coating. In still other embodiments, the therapeutic agent is a chemotherapeutic drug and the target cell or tissue is of brain cancer.

Also encompassed by the invention are methods of treating diseases, particularly diseases associated with brain tissue, the methods comprising: administering a therapeutically effective amount of a therapeutic agent to an individual having the disease, the therapeutic agent being loaded onto lipid nanoparticles comprising: a lipid core comprised of a triglyceride component and a cholesterol ester component; a phospholipid layer; a surfactant coating surrounding the phospholipid and the lipid core; and a human recombinant apolipoprotein (ApoE3) adsorbed to a surface of the nanoparticle without Polysorbate 80, wherein the apolipoprotein is human recombinant ApoE3, wherein the therapeutic agent is transported in the lipid nanoparticle across the blood-brain barrier to the target brain tissue through transcytosis independent of LDL receptor binding.

In embodiments, the therapeutic agent may be an antibiotic and the disease is an intracerebral infection of Candida albicans. In certain embodiments, the antibiotic is Amphotericin B. According to certain methods of the invention, the therapeutic agent is Amphotericin B that has at least 40% less toxicity in human red blood cells than a conventional formulation of Amphotericin B having a similar Minimum Inhibitory Concentration. For example, the therapeutic agent may have at least 50% less toxicity or even at least 60% less toxicity in human red blood cells than if administered in a conventional formulation.

In some treatment methods, the therapeutic agent may also be a diagnostic magnetic resonance imaging contrast agent, such as one selected from Gadolinium-, Magnetite-, and Fluorophore-based contrast agents. In some embodiments, the therapeutic agent is a chemotherapeutic drug for treatment of brain cancers. The lipid nanoparticles loaded with the therapeutic agent may be administered in a pharmaceutical composition comprising the lipid nanoparticles and a pharmaceutically acceptable excipient. Such administration is preferably selected from intravenous or intranasal.

In still other embodiments, the invention provides for methods of treating skin conditions associated with reduced collagen production, comprising topically applying a composition comprising a therapeutically effective amount of lipid nanoparticles to an affected area on a surface of the skin, the lipid nanoparticles comprising: a lipid core comprised of a triglyceride component and a cholesterol ester component; a phospholipid layer; a surfactant coating layer surrounding the phospholipid layer and the lipid core; a human recombinant apolipoprotein (ApoE3) bonded to a surface of the nanoparticle without Polysorbate 80; and at least one therapeutic agent in the lipid core, wherein the nanoparticles diffuse from the surface of the skin across the epidermis, resulting in the therapeutic agent being intracellularly released in the dermis by LDL receptor-mediated endocytosis and stimulating fibroblast collagen production.

In the methods of treating skin conditions, the composition may be in the form of a cream or a gel. In certain embodiments, the therapeutic agent is Retinoin. In other embodiments, the therapeutic agent is Ingenol.

### BRIEF DESCRIPTION OF THE DRAWINGS

FIG. 1 is an illustration of an exemplified configuration of the lipid nanoparticle used in certain embodiments of the invention.

FIGS. 2A-2B show capillary electrophoresis in MECC conditions for: (FIG. 2A) a nanoparticle (peaks 2, 3, 4); and (FIG. 2B) a nanoparticle with ApoE3rec standard added at 2.185 mg/ml (peak 1). MECC conditions: capillary, 50 mm ID, 60 cm length. Running buffer: 16 mM boric acid, 40 mM SDS, pH 7.0. Sample preparation: diluted 2/200 in running buffer for analysis, 25 kV, normal polarity, 25 minutes. Addition of the standard: diluted 20/220.

FIG. 3 shows uptake of lipid nanoparticles that have been targeted with ApoE3 and the same nanoparticles without ApoE3, expressed as moles of Gd normalized to mg of cell proteins.

FIG. 4 shows an MRI image of cells incubated with Gadolinium lipid nanoparticles with and without ApoE3.

FIG. 5A shows signal intensity as measured on the total cerebral tissue after injection of lipid nanoparticles targeted with ApoE3 and lipid nanoparticles without ApoE3. FIGS. 5B and 5C represent the T1-weighted brain images, with red grayscale pixels showing a SI increase by >3 SD of the pre-contrast tumor.

FIG. 6A shows the volume distribution and average size of lipid nanoparticles for a formulation of N416, and FIG. 6B shows the volume distribution and average size of lipid nanoparticles for a formulation of N436.

FIGS. 7A-7D show quantitative uptake profiles for a nanoparticle loaded with DiR used in methods of the invention, wherein the nanoparticle with ApoE3 is captured in the liver (FIG. 7A), brain (FIG. 7B), lung (FIG. 7C), and kidney (FIG. 7D). The uptake profile for corresponding nanoparticles without ApoE3 is also shown for the respective tissues evaluated.

FIG. 8 shows the volume distribution and average size of an ApoE3 lipid nanoparticle with Magnetite in formulation N381.

### DETAILED DESCRIPTION OF EMBODIMENTS OF THE INVENTION

The present invention may be understood more readily by reference to the following detailed description of the preferred embodiments of the invention. However, although different components and methods are disclosed and described, it is to be understood that this invention is not limited to specific formulations, assemblies or configurations, conditions, or methods, as such may vary, and any modifications thereto and variations therein will be apparent to those skilled in the art. It is also to be understood that the terminology used herein is for the purpose of describing specific embodiments only and is not intended to be limiting.

### I. Definitions

It must be noted that as used herein and in the appended claims, the singular forms “a,” “an,” and “the,” include plural forms unless the context clearly indicates otherwise. Thus, for example, reference to “a nanoparticle” or “a therapeutic agent” includes one or more of such same or different nanoparticles or therapeutic agents, respectively. Reference to “the method” includes reference to equivalent steps and methods known to those of ordinary skill in the art that could be modified or substituted for the methods described herein.

As used herein, the term “administering” refers to the placement of the lipid nanoparticles loaded with therapeutic agent into a subject by a method or route which results in at least partial localization of the therapeutic agent(s) at a desired site. The nanoparticles with therapeutic agent(s) can be administered in any suitable form and by any appropriate route that results in effective treatment in the subject.

As used herein, the term “LDL receptor” refers to a low density lipoprotein receptor family that comprises at least 10 members in mammals: the LDL receptor (LDLr) itself, the apolipoprotein E receptor (ApoER2), the very low density lipoprotein receptor (VLDLr), the LDL related receptor (LRP), LRP1B, megalin, LRP3, LRP4, LRP5, and LRP6.

As used herein, the term “lipid binding protein” means a protein which may be associated with the phospholipids monolayer of the nanoparticle, preferably an apolipoprotein, including (but not limited to) ApoA, ApoB, ApoC, ApoD, ApoE, and all isoforms of each. As used herein, the term “ApoE” means one or more of the isoforms of ApoE, including but not limited to ApoE2, ApoE3, and ApoE4. In preferred embodiments of the invention, ApoE3 is used as the apolipoprotein of the lipid nanoparticles.

“Controlled release” as used herein refers to release of a therapeutic agent from the nanoparticle so that the blood or tissue levels of the pharmaceutically active ingredient thereof, or of the therapeutic agent, is maintained within a desired therapeutic range for an extended period (hours or days).

“Nanoparticles” are particles with a diameter of less than about 1,000 nm (1 μm) comprising various biodegradable or non-biodegradable polymers, lipids, phospholipids or metals. (See Jin, Y., Nanotechnology in Pharmaceutical Manufacturing, Pharmaceutical Manufacturing Handbook: Production and Processes. Vol. 5, Section 7, John Wiley & Sons (2000); and Lockman, P. R. et al., “Nanoparticle technology for drug delivery across the blood-brain barrier,” Drug Development and Industrial Pharmacy 28.1: 1-13 (2002)). The nanoparticles employed in the methods of the invention, and methods for their manufacture, are described in U.S. patent application Ser. No. 15/760,170 (incorporated herein by reference) and specifically include ApoE3.

“Nanoemulsion” as used herein refers to a nanosized colloidal systems that consists of poorly water soluble compounds, suspended in an appropriate dispersion medium (oil-in-water emulsion) stabilized by surfactants.

As used herein, the terms “therapeutic agent,” “active agent” or “active ingredient” means therapeutically useful amino acids, peptides, proteins, nucleic acids, including but not limited to polynucleotides, oligonucleotides, genes and the like, carbohydrates and lipids. The therapeutic agents according to embodiments of the invention may include neurotrophic factors, growth factors, enzymes, antibodies, neurotransmitters, neuromodulators, antibiotics, antiviral agents, antifungal agents and chemotherapeutic agents, and the like. The therapeutic agents of the present invention include drugs, prodrugs, antibiotics, diagnostic substances, contrast agents and precursors that can be activated when the therapeutic agent is delivered to a target cell or tissue.

As used herein, the term “pharmaceutically acceptable carrier” means a chemical composition or compound with which an active ingredient may be combined and which, following the combination, can be used to administer the active ingredient to a patient. In embodiments, “pharmaceutically acceptable carrier” also includes, but is not limited to, one or more of the following: excipients, surface active agents, dispersing agents, inert diluents, granulating and disintegrating agents, binding agents, lubricating agents, sweetening agents, flavoring agents, coloring agents, preservatives, physiologically degradable compositions such as gelatin, aqueous vehicles and solvents, oily vehicles and solvents, suspending agents, dispersing or wetting agents, emulsifying agents, demulcents, buffers, salts, thickening agents, fillers, antioxidants, stabilizing agents, and pharmaceutically acceptable polymeric or hydrophobic materials.

As used herein, “an effective amount” refers to the amount sufficient to bring about a desired result in an experimental setting. A “therapeutically effective amount” or “therapeutic dose” refers to an amount sufficient to produce a therapeutic response or beneficial clinical result in a patient. For the methods of the invention, the therapeutically effective amount or dose can be estimated initially from cell culture assays, then the dosage can be formulated for use in animal models so as to achieve a circulating concentration range that includes the IC₅₀ as determined in cell culture. Such information can then be used to more accurately determine useful doses in humans.

As used here in the term “minimum inhibitory concentration” or “MIC” refers to the lowest concentration of a chemical compound/substance which prevents visible growth of a microorganism.

As used herein, the terms “patient” and “individual” refer to any person or other subject is in need of, and would receive a benefit from, administration of the lipid nanoparticles according to therapeutic methods described herein. It is envisioned that the “patient” may also be a non-human animal, such as, e.g., in veterinary applications of the invention.

As used herein, the term “Selectivity Index” or “SI” refers to a comparison or ratio between the IC₅₀ in healthy cells and the IC₅₀ in diseased cells. The SI value shows the differential activity of a product between healthy and non-healthy cells. The higher the value, the more selective the product will be.

As used herein, the term “therapeutic index” or “TI” refers to a comparison or ratio of the amount of a therapeutic agent that causes the therapeutic effect to the amount that causes toxicity, and is calculated as TI=LD₅₀/ED₅₀ (lethal dose 50/effective dose 50).

As used herein, the term “therapeutic window” refers to the range of a drug's dosage or serum concentration at which a desired effect occurs in a bodily system. For example, there is typically little or insufficient effect below the therapeutic window, whereas toxicity could occur above the therapeutic window range.

### II. Lipid Nanoparticles Used in Methods of the Invention

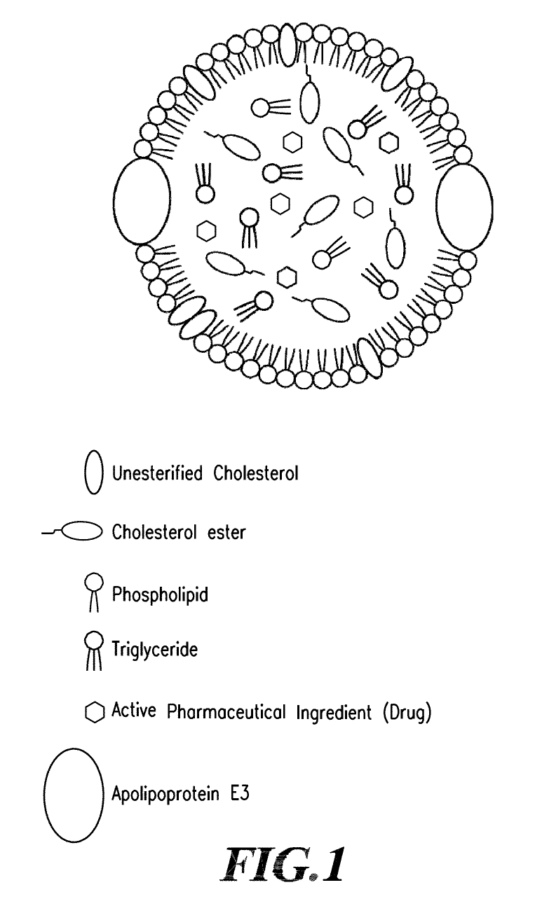

The delivery or carrier mechanism in the methods of the invention is an improved lipid nanoparticle, as described in U.S. patent application Ser. No. 15/760,170 (incorporated herein by reference). The structure/configuration of a lipid nanoparticle according to certain embodiments is depicted in FIG. 1. The ingredients are distributed so as to form a lipid core, covered by a phospholipid layer, and finally a surfactant coating layer. The therapeutic agent, or active pharmaceutical ingredient, is located in the lipid core and/or the phospholipid layer; and a lipid binding protein (e.g., ApoE3) is bonded to the surface of the nanoparticle. Notably, the apolipoprotein is bonded without Polysorbate 80.

The structure and behavior of nanoparticles are consequences of their composition. In the lipid nanoparticles used in the methods of the invention, the specific composition of ingredients (as described in U.S. patent application Ser. No. 15/760,170) results in an improved and stable nanoparticle having structural characteristics desirable for drug delivery.

There are natural occurring complex particles in plasma with a central core containing cholesterol esters and triglycerides surrounded by free cholesterol, phospholipids and apolipoproteins. The lipoproteins are classified based on size, lipid composition, and apolipoproteins: chylomicrons, VLDL (very low density lipoproteins), IDL (intermediate density lipoproteins), LDL (low density lipoproteins), HDL (high density lipoproteins).

Even though the nanoparticles employed herein contain lipid binding protein ApoE3, which is typical component of LDL, LDLs are defined to have a diameter of about 20-25 nm, a density of 1.019-1.063 g/ml, and comprised of about 21-25% proteins and 79-75% lipids. Thus, the nanoparticles employed in the methods of the invention would not be considered to be artificial LDLs, since their average size is larger than a typical LDL, and the concentration ranges and resulting ratios of the respective components are also different from that of natural LDL particles.

In the preferred nanoparticles employed in the invention, the lipid core of the nanoparticle is non-aqueous and has a high retention capacity for the lipophilic (or liposoluble) active ingredient(s). The lipid binding protein is preferably an apolipoprotein, such as ApoE3 or analogs thereof. In preferred embodiments, the apolipoprotein is recombinant ApoE3 and may be further modified to enhance targeting efficacy of the active ingredient(s). The lipid nanoparticles may be spherical, oval, or discoid in shape and have a diameter of about 20-150 nm, such as 30-120 nm, or 50-100 nm.

Lipids suitable for use in nanoparticles of the invention include (but are not limited to) phospholipids, triacylglycerols, cholesterol, cholesterol esters, fatty-acyl esters, and the like. Preferably, nanoparticles of the invention are generally formed of the following five components: (1) phospholipid, (2) triglyceride, (3) cholesterol ester, (4) cholesterol, and (5) ApoE3. For example, in a preferred embodiment, the lipid core may be made of cholesterol ester and triglyceride (e.g., castor oil), the phospholipid layer may be made of egg yolk phospholipid, and the surfactant coating layer may be made of sodium taurodeoxicholate and Poloxamer 188.

A. Phospholipids of the Nanoparticle

Phospholipids suitable for use in the nanoparticles include (but are not limited to) diacylglyceride structures and phosphophingolipids. Diacylglycerides structures include phosphatidic acid (phosphatidate) (PA); phosphatidylethanolamine (cephalin) (PE), phosphatidylcholine (lecithin) (PC), phosphatidylserine (PS) and phosphoinitides. The phosphosphingolipids include ceramide phosphorylcholine (Sphingomyelin) (SPH), ceramide phosphorylethanolamine (Sphingomyelin) (Cer-PE) and ceramide phosphoryl lipid. The phospholipids suitable for use in the nanoparticles formulation include natural phospholipid derivatives and synthetic phospholipid derivatives. Natural phospholipid derivatives include egg PC, egg PG, soy PC, hydrogenated soy PC and sphingomyelin. Synthetic phospholipid derivatives include: phosphatidic acid; phosphatidylcholine; 1,2-didecanoyl-sn-glycero-3-phosphocholine (DDPC); 1,2-Dilauroyl-sn-glycero-3-phosphocholine (DLPC); 1,2-dimyristoyl-sn-glycero-3-phosphocholine (DMPC); 1,2-Dipalmitoyl-sn-glycero-3-phosphocholine (DPPC); 1,2-Distearoyl-sn-glycero-3-phosphocholine (DSPC); 1,2-Dioleoyl-sn-glycero-3 -phosphocholine (DSPC); 1-Palmitoyl-2-oleoyl-sn-glycero-3-phosphocholine(POPC); 1,2-Dierucoyl-sn-glycero-3-phosphocholine (DEPC); phosphatidylglycerol (DMPG); 1,2-Dimyristoyl-sn-glycero-3-phosphoalycerol; 1,2-Dipalmitoyl-sn-glycero-3-phosphoglycerol (DPPG); 1,2-Distearoyl-sn-glycero-3-phosphoglycerol (DSPG); 1-palmitoyl-2-oleoyl-sn-glycero-3-phosphoglycerol (POPG); Phosphatidtylethanolamine(DMPE); 1,2-Dimyristoyl-sn-glycero-3-phosphoethanolamine; 1,2-dipalmitoyl-sn-glycero-3-phosphoethanolamine (DPPE), 1,2-Distearoyl-sn-glycero-3-phosphoethanolamine (DSPE); 1,2-Dioleoyl-sn-glycero-3-phosphoethanolamine (DOPE).

In an embodiment, phospholipids suitable for use in the nanoparticles comprise 1,2-dimyristoyl-sn-glycero-3-phosphocholine (DMPC); phosphatidyl glycerol (DMPG);1,2-distearoyl-sn-glycero-3-phosphocholine (DSPC): 1,2-distearoyl-sn-glycero-3-phosphoglycerol (DSPG); and egg PC. In one embodiment, the phospholipid is egg PC.

B. Triglycerides of the Nanoparticle

Triglycerides suitable for use in the nanoparticles formulation include (but are not limited to) triglycerides which are liquid at room temperature. Triglycerides suitable for use in the nanoparticles are selected from the group comprising canola oil, castor oil, chia seed oil, coconut oil, corn oil, cottonseed oil, olive oil, palm oil, peanut oil, safflower oil, sesame oil, soybean oil and others. Triglycerides also include mono-, di- and tri-acyl glycerols, where the fatty acids can be Mono-unsaturated fatty acid (palmitoleic acid, oleic acid, elaidic acid, gadoleic acid, eicosenoic acid, erucic acid and others), di-unsaturated fatty acid (linoleic acid, eicosadienoic acid, docosadienoic acid and others) and polyunsaturated fatty acids (linolenic acid, dihomo-γ-linolenic acid, eicosatrienoic acid, stearidonic acid, arachidonic acid, eicosatetraenoic acid, eicosapentaenoic acid, tetracosanolpentaenoic acid, docosahexaenoic acid and others). The di- and tri-acyl glycerols can contain or not identical fatty acids. Fractionated triglycerides, modified triglycerides, synthetic triglycerides, hydrogenated triglycerides and mixtures of triglycerides are also within the scope of the invention and mixtures thereof.

In embodiments, triglycerides suitable for use in the nanoparticles comprise castor oil, soy oil, coconut oil, and/or hydrogenated castor oil. In certain embodiments, the triglyceride of the nanoparticles is castor oil, and the therapeutic agent may be dissolved in this component within the nanoparticle core.

C. Cholesterol and Cholesterol Esters of the Nanoparticle

Cholesterol esters refer to cholesterol esterified with saturated fatty acid, including (but not limited to) myristic acid, palmitic acid, stearic acid, arachidic acid, lignoceric acid, and the like, or an unsaturated fatty acid, including but not limited to palmitoleic acid, oleic acid, vaccinic acid, linoleic acid, linolenic acid, arachidonic acid, eicosatrienoic acid, stearidonic acid, arachidonic acid, eicosatetraenoic acid, eicosapentaenoic acid, tetracosanolpentaenoic acid, docosahexaenoic acid and the like.

In some embodiments, the cholesterol ester of the nanoparticles is cholesteryl oleate. The cholesterol esters are located in the lipid core, whereas cholesterol is located in the phospholipid layer. Cholesterol is typically used in a proportion of between 0 and 4%, or in at least 0.1%, at least 0.5%, or at least 1%, and up to 3.9%, or up to 3.5%, or up to 3% of the nanoparticle components.

D. Lipid Binding Protein of the Nanoparticle: Apolipoproteins

In compositions of the invention, the surface of the nanoparticles has bonded the lipid binding protein, preferably an apolipoprotein such as ApoE3. The apoprotein molecule is responsible for binding to lipoprotein receptors in the targeted tissues. According to Mims et al., Biochemistry 29(28):6639-47 (1990), depending on the state of the lipid constituents, the apoproteins undergo structural changes.

As previously mentioned, the main groups of lipoproteins are classified as chylomicrons, very-low-density lipoproteins (VLDL), low-density lipoproteins (LDL), and high-density lipoproteins (HDL) based on the relative densities of the aggregates on ultracentrifugation and with fortuitously broadly distinct functions. These classes can be further refined by improved separation procedures, and each may have distinctive apoprotein compositions and biological properties. Density is determined largely by the relative concentrations of triacylglycerols (lighter) and proteins, and by the diameters of the broadly spherical particles. The data for the relative compositions of the various lipid components in the natural lipoparticles should not be considered as absolute, as they are in a state of constant flux. In general, however, the lower the density class, the higher the proportion of triacylglycerols and the lower the proportions of phospholipids and the other lipid classes. In fact, the VLDL and LDL exhibit a continuum of decreasing size and density.

Lipids generally comprise about 75% (75%-79%) of the mass of the LDL particle, and proteins generally make up about 25% (21%-25%). Furthermore, the lipid component of LDL consists primarily of an apolar core of neutral lipid comprised mostly of esterified cholesterol. Surrounding this apolar core is a lipid coat composed of phospholipids and free cholesterol. Of the total lipid in the LDL particle, cholesterol comprises 60%, of which approximately 80% is in the form of cholesteryl esters in the core of the particle. The major fatty acid of the cholesteryl esters of LDL is linoleate, which accounts for 50% of the total, with oleate and palmitate comprising 20% and 15% of the cholesteryl ester fatty acids, respectively. The phospholipids of LDL, which comprise 30% of its total lipid, consist primarily of phosphatidylcholine (65%) and sphingomyelin (25%). (See Joseph L. Goldstein and Michael S. Brown, “The Low-Density Lipoprotein Pathway and Its Relation to Atherosclerosis,” Ann. Rev. Biochem. 46:897-930 (1997)).

In this regard, while Nelson et al. describe nanoparticles with a preferred density between about 1.00 and 1.07 g/ml, where the phospholipids and lipids are added in a ratio of between 11.5:1 and 12.5:1; nanoparticles with a diameter of between 10 and 50 nm are obtained, which nanoparticles can easily be considered as artificial LDLs; the nanoparticles employed in the inventive methods have an average size of 20-150 nm along with the concentration ranges of the components that do not correspond to the LDL description. The presence of these particular ingredients in this specific proportions results in an improved nanoparticle with desirable characteristics.

As shown in Table 1 below, the charge capacity of these synthetic LDLs (Nelson et al.) is only 10% greater than the particles according to an exemplified embodiment of the invention. Furthermore, Nelson achieves the desired loading capacity by conjugating the active ingredient with cholesterol. In contrast, no covalent bond is needed for loading the nanoparticles employed by the methods of the invention.

TABLE 1

| | | | | Nanoparticle |
|---|---|---|---|---|
| | | | | (described in |
| | | | | U.S. Patent |
| | | | | Application |
| | Nelson et | | | No. |
| | al. | McChesney | Plasma LDL | 15/760,170) |
| Phospholipids | 84% | 78% | 20-28% | 37% |
| Triglycerides | — | 10% | 10-15% | 56% |
| Cholesterol | — | 2% | 37-48% | 1% |
| Esters | | | | |
| Cholesterol | 4% | 1% | 8-10% | 2% |
| Proteins (Apo E) | 8% | — | 20-22% | 1% |
| Active Ingredient/ | 4% | 9% | — | 3% |
| Therapeutic | | | | |
| Agent | | | | |
| Particles Size | 10-50 nm | 40-80 nm | 20-25 nm | 20-150 nm |

ApoE is an apoprotein involved in cholesterol transport and plasma lipoprotein metabolism throughout the body. In peripheral cells, ApoE influences cellular concentrations of cholesterol by directing its transport. In neurons, changes in cholesterol levels influence the phosphorylation status of the microtubule-associated protein at the same sites that are altered in Alzheimer's disease. This apoprotein has three major isoforms: ApoE4, ApoE3, and ApoE2, differing by single amino acid substitutions. At physiological concentrations (micromolar), ApoE exists predominantly as a tetramer. In a lipid-free state, the carboxy-terminal domain of the apolipoprotein forms a dimer, which then dimerizes to form the tetramer. However, ApoE is likely to bind to lipids in its monomeric, rather than tetrameric, state. (See Hatters et al., “Apolipoprotein E Structure: Insights Into Function,” Journal of Biological Sciences, 31(8), 445-454 (2006); and Peters-Libeu et al., “Model of Biologically Active Apolipoprotein E Bound to Dipalmitoylphosphatidylcholine,” Journal of biological Chemistry 281(2), 1073-79 (2006)).

In preferred embodiment, nanoparticles administered according to methods of the invention comprise ApoE3 as the apolipoprotein component. Preferably, the nanoparticles comprise recombinant or cloned ApoE3 which may be further modified to enhance targeting efficacy. The use of recombinant ApoE3 avoids problems with antigenicity due to possible post-translationally modified, variant, or impure ApoE3 protein purified from human donors.

McChesney et al. described synthetic LDL prepared with any protein wherein the nanoparticle becomes coated with native apolipoprotein upon intravenous injection and is recognized and internalized by cellular LDL receptors. In this regard, and as previously stated, there is information showing that each individual has different levels of Apo proteins in the body, and these levels also vary depending on the physiological conditions. (See Liuet al., 2015; Vila-Rodriguez et al., 2001; Robitaille et al., 1996; Valdez et al., 1995; Haffner et al., 1996; Utermann et al., 1987). Thus, depending on the amount of Apo proteins available and the predominant isoform in each individual, this can result in a large variability of the results, which is not desirable for a pharmaceutical composition and therapeutic uses.

In embodiments of the invention, the recombinant ApoE3 has a high affinity for the exposed surface of the nanoparticles and therefore sticks to the nanoparticles under the specific conditions discussed in connection with the manufacturing method. The average size of the ApoE3 is about 10.67±2.02 nm (n=4, media±SEM), and a Z-potential of the ApoE3 is about −14.47±1.18 mV (n=4, media±SEM), at pH 7.4.

Although the specific lipid components stated above may be preferred, embodiments of the invention may include other lipids, for example to include chemically-modified lipids, or admixtures of other naturally occurring lipophilic molecules that may work equally well. Persons skilled in the art will understand that modifications may be made to adapt the nanoparticles for a specific therapeutic agent or therapeutic application.

ApoE3 may be contained in the nanoparticles in an amount as low as 1% or less and does not require Polysorbate 80 for adhesion to the surface. In preferred embodiments, the nanoparticles do not contain any Polysorbate 80.

E. Therapeutic Agents

The nanoparticles employed in the methods of the invention described herein comprise one or more therapeutic agents, as described further below in connection with specific methods of the invention.

The therapeutic agent, or lipophilic active ingredient(s), are encapsulated by the nanoparticles, and preferably dissolved in the triglyceride component. Notably, no covalent modification of the therapeutic agent is required for incorporation in the nanoparticles. In preferred embodiments, the therapeutic agent is not conjugated with another molecule within the core. That is, the lipid core of the nanoparticles has high retention capacity for liposoluble active ingredients without the need for conjugation. This is yet another advantage of the nanoparticle and the manufacturing process thereof according to embodiments of the invention, as there is evidence showing differences in activity between conjugated and non-conjugated therapeutic agents. For instance, there is evidence suggesting decreased activity of some drugs when the therapeutic agent is conjugated. There are results that for Paclitaxel bonded to oleic acid, the IC₅₀ increases 10-fold compared with free drug, meaning that it takes 10 times more conjugated drug to produce the same effect than the drug in free form. (See Feng, Lan et al., 2011: Lundberg B. et al., 2003: Rodrigues, G. et al., 2005). Moreover, conjugation of a therapeutic agent requires a chemical reaction, or at least one additional step during the manufacturing process, which—as discussed in U.S. patent application Ser. No. 15/760,170—is not needed in the preparation/manufacture of these nanoparticles.

The therapeutic agent(s) can be associated with the nanoparticle by any method known to the skilled artisan, including preferably encapsulation in the interior or association with the lipid portion of the nanoparticle

The amount of therapeutic agent present in the nanoparticles will vary in different embodiments of the invention, particularly depending on the therapeutic agent used. However, for optimal incorporation into the nanoparticle, the amount of therapeutic agent should be 1 gram drug per 20-40 grams of lipids (total lipid content); or 1 grain of drug per 10-25 grams of triglycerides; or 1 gram of drug per 7-15 grams of phospholipids. Multiple therapeutic agents or additional agents may be present in the core of the same particle, depending on the desired therapeutic objective.

The therapeutic agent can be any desired entity, e.g., polypeptide, polynucleotide, chemical compound, growth factor, hormone, antibody, cytokine, or the like, including those entities that cannot otherwise pass across the blood-brain barrier by themselves (in conventional or free form).

A wide variety of therapeutic agents are available and encompassed by methods of the present invention. For example, therapeutic agents according to various embodiments of the invention include, but are not limited to, chemotherapeutic agents for treating brain tumors with agents that do not reach the tumor in sufficient amounts when tolerable doses are administered systemically in conventional form; and antibiotics for treating infectious diseases, especially where penetration into the brain of such systemically administered antibiotics is otherwise a block to treatment.

In some embodiments, the therapeutic agent can be a diagnostic agent, such as an imaging agent and, in particular, contrast media for brain imaging that are currently not used because of poor penetration into the brain upon systemic administration (delivery in free form). Diagnostic agents suitable for use in molecular diagnostic procedures include, e.g., positron-emission tomography (PET), computed tomography (CT) or ultrasound, and magnetic resonance imaging (MRI), and optical imaging techniques (both fluorescence and near infrared (NiR)). Of these techniques, MRI has not been applied to its full potential due to its low specificity. However, the lack of MRI specificity can be improved using cell markers and the properties of paramagnetic and superparamagnetic particles, which can be utilized for detection in small quantities with MRI.

Contrast enhancement can be provided by, e.g., Gadolinium, Magnetite, Fluorescein, 5 aminolevulinic acid, lipophilic tracers (DiI, DiO, DiD, DiA, and DiR), methylene blue, and/or indocyanine green. Delivery of such diagnostic agents (as therapeutic agent(s) of the invention) can enhance the imaging of brain tissue structures and function. In certain embodiments, the therapeutic agent may be an agent for diagnosis for cancer, and/or of brain diseases or associated conditions.

As one object of the invention, provided are targeted delivery methods of drugs that are highly toxic for human tissue, such as, e.g., cancer treatment drugs. In embodiments, the therapeutic agent is a lipophilic drug and preferably a chemotherapeutic drug. The term “chemotherapeutic drug” is used to refer to an agent that can be used in the treatment of cancers, for example brain cancers and gliomas and that is capable of treating such cancers. In some embodiments, a chemotherapeutic agent can be in the form of a prodrug which can be activated to a cytotoxic form. Conventional chemotherapeutic agents that are known by persons of ordinary skill in the art are encompassed for use in method of the present invention. For example, chemotherapeutic drugs for the treatment of brain tumors and gliomas include, but are not limited to: temozolomide, procarbazine, and lomustine. Chemotherapeutic agents given intravenously include vincristine, cisplatin, carmustine, carboplatin, and mexotrexate. In certain embodiments, a chemotherapeutic agent may include taxane, abeo-taxane, and other molecules derived from taxanes. In certain embodiments, the chemotherapeutic agent may include, e.g., paclitaxel, docetaxel, cabazitaxel, and the like.

As another object of the invention, provided are delivery methods of therapeutic agents that are useful for treating brain diseases and/or associated conditions. For example, a therapeutic agent to be delivered by the methods disclosed herein can be a pharmaceutically active agent that at least as part of its action targets the central nervous system, olfactory system, visual system, or any other system associated with brain disorders.

In embodiments, the therapeutic agent can be transported to various target cells or tissues across the blood-brain barrier and have preferential uptake in the brain, lung, kidneys, and liver. In some embodiments, the therapeutic agents are cytotoxic or growth-suppressing polypeptides that can be used inside the blood-brain barrier to treat certain types of cancer or other disease. Therapeutic agents useful in the present invention include various types of receptor antagonists, antibodies, and other polypeptides that can block or suppress one or more types of neuronal activity and can be used to help control and reduce neuropathic pain, hyperalgesia, and similar problems.

As still another object of the invention, provided are methods for treatment of skin conditions associated reduced collaged production. Although it is conventionally known that topical application of tretinoin (retinoic acid) improves fine wrinkles associated with damage caused by exposure to sunlight (photodamage), it is also believed that reduction of collagen levels in areas of the skin exposed to the sun is an etiological component.

Topical treatment of acne vulgaris and dermatoheliosis (photodamage) was originally performed with RETIN-A (topical tretinoin) in gel or cream form, stimulating the production of new non-adherent corneal cells within the follicular canal and accelerating the detachment of old cells from the superficial layers up to 6 times the normal rate of velocity.

RETIN-A micro gel beads, loaded with tretinoin at 0.1%, 0.08% and 0.04% is a new and superior product to the traditional RETIN-A gel or cream because it does not expose the skin to a high concentration of tretinoin and reduces its side effects of erythema, peeling, itching and burning. This is due to the gradual release of tretinoin which avoids delivery of a high concentration of the active substance.

In certain embodiments of the invention, the therapeutic agent is tretinoin. By this therapeutic agent being loaded into the lipid nanoparticle, it will avoid the undesired surface contact with the skin (due to it being within the ApoE-modified lipid nanoparticle) upon administration, and, as a result, will reduce the side effects of the conventional microspheres of RETIN-A gel.

In another embodiment, the invention relates to treatment of non-hypertrophic actinic keratosis in adults. In such embodiments, the therapeutic agent loaded on the ApoE-modified lipid nanoparticle is Ingenol. Ingenol is a molecule that binds and activates protein kinase C and induces similar responses to phorbol esters in biological systems. Concentration values of Ingenol according to embodiments of the invention range between 30 uM and 1 mM.

### III. Lipid Nanoparticle Properties and Characteristics

As described in application Ser. No. 15/760,170, the present inventors have discovered that the presence of the five component types described above, in specific concentrations, results in the nanoparticles having the desirable characteristics described and in connection with the methods of the invention. That is, the specific concentration ratios of the respective components, as well as the presence of ApoE3, are critical to achieving the advantageous and unexpected results of the nanoparticles, as compared to conventional nanoparticle formulations.

Specifically, the concentration ranges for the respective components, and the resulting ratios thereof, have been found to have an unexpected and synergistic effect. Summarized in Tables 2 and 3 below are preferred concentration content ranges (% w/w), and the optimal ratios thereof, of the respective components of the nanoparticles without cryopreservants or salts.

In the prior U.S. patent application Ser. No. 15/760,170, the nanoparticles are described as comprising the therapeutic agent Docetaxel and ApoE3 in a molar ratio of from 45-140 (ratio of molecules of Docetaxel per each recombinant ApoE3 molecule). A mass ratio of Docetaxel (therapeutic agent) to ApoE3 in the nanoparticles is preferably from 1.1 to 3.3 (Docetaxel to ApoE). Substantially similar or the same ratios correspond to the content ratios of therapeutic agent(s) (other than Docetaxel) described herein for administration according to methods of the invention, or encompassed by the scope of the present disclosure.

TABLE 2
Content Ranges (% w/w) of Nanoparticle Components

| | | Cholesterol | | |
|---|---|---|---|---|
| Phospholipids | Triglycerides | Ester | Cholesterol | Apo E3 |
| 2.25-8.25 | 3.75-12.1 | 0-0.6 | 0-0.9 | 0.1-01.4 |

| Phos- | | Cholesterol | Cho- | | Therapeutic |
|---|---|---|---|---|---|
| pholipids | Triglycerides | Ester | lesterol | Apo E3 | Agent (drug) |
| 5.25-8.25 | 3.75-12.1 | 0-0.6 | 0-0.9 | 0.1-1.4 | 0.3-0.9 |

TABLE 3
Optimal Ratios of Nanoparticle Components

| | | Cholesterol | | |
|---|---|---|---|---|
| Phospholipids | Triglycerides | Ester | Cholesterol | Apo E3 |
| 35-38 | 25-60 | 0-3 | 0-4 | 0.5-7 |

| Phos- | Tri- | Cholesterol | | | Active |
|---|---|---|---|---|---|
| pholipids | glycerides | Ester | Cholesterol | Apo E | Ingredient |
| 35-38 | 26-60 | 0-3 | 0-4 | 0.5-7 | 1-10 |

It has been found that, in the nanoparticles, phospholipid/triglyceride ratios between 0.58 and 6.4 are convenient. The phospholipid and triglyceride components are preferably present in the nanoparticle in a ratio ranging from 5.25-8.27 (phospholipids) to 3.75-12.1 (triglycerides). The ratio PL/TG between 0.58 and 0.78 is helpful for maximum loading capacity of the nanoparticles. Also, nanoparticles with a preferred PL/TG ratio (e.g., 0.67) and free cholesterol (PL: TG: EC: CL) of 39:58:1:2 are the ones that results in the highest loading capacity (percentage of encapsulation efficiency) for the active ingredient (therapeutic agent). Furthermore, the weight ratio of the phospholipid and triglyceride components provides a therapeutic agent encapsulation efficiency of the nanoparticles of at least 80%, preferably at least 85%, and even more preferably at least 90%, as determined by HPLC.

As demonstrated in the associated application disclosure, the combination of components in specific content ratios lead to synergistic results with respect to the advantageous properties (e.g., loading capacity, encapsulation efficiency) of the nanoparticles. Furthermore, as evidenced by the various Examples included in U.S. patent application Ser. No. 15/760,170, the above-described contents and ratios of the nanoparticle components are critical to achieving the unexpected characteristics and properties of the nanoparticles which are used and applied to the methods of the invention described herein.

For example, it was found that varying the ratio of phospholipids/triglycerides results in changes to the charging efficiency of the nanoparticles. Specifically, lipid nanoparticles with a phospholipid/triglyceride ratio in the aforementioned ratio range exhibited the highest percentage of encapsulation efficiency for the active ingredient (85+5%). (This was determined by HPLC and based on the % of drug that was released from the nanoparticle.) Additionally, the lipid nanoparticles comprising ApoE3 demonstrated modified zeta potentials without any significant changes to the nanoparticle size (FIGS. 12 and 13 of U.S. patent application Ser. No. 15/760,170). As also demonstrated by FIG. 9 of that application, lipid nanoparticles with the same concentration for the respective components but with variations in the nature of employed triglyceride show differences both in the Z-average of the nanoparticles and dispersion (Pdi). The nanoparticles made with castor oil result in smaller particle size. Furthermore, nanoparticles prepared with castor oil result on a more defined form (less amorphous) that can be deduced from the minor difference between the Z-average and Volume values.

A fundamental characteristic of nanoparticles is their instability. As particle size goes down, the interfacial area per unit mass of the dispersed system increases, and so does the interfacial energy. This increased energy will tend to drive the particles to coalescence, forming larger particles with lower energy. Extreme particle size reduction can result in significant increases in drug solubility. Materials in a nanoparticle have a much higher tendency to leave the particle and go into the surrounding solution than those in a larger particle of the same composition. This phenomenon can increase the availability of drug for transport across a biological membrane, but it can also create physical instability of the nanoparticle itself. This instability is seen in Ostwald ripening in which small particles disappear as material is transferred to large particles. The physical stability of nanoparticles may be improved by the use of appropriate surface active agents and excipients at the right levels to reduce the interfacial energy, controlling the surface charge of the particles to maintain the dispersion, and manufacturing the particles in a narrow size distribution to reduce Ostwald ripening.

The nanoparticles employed in the inventive methods preferably have an average size between 20 and 150 nm, such as between 50 and 120, a Z potential between −25 and −5 mV, and a PDI Dispersion Value between 0.08 and 0.30. In a culture with lipoprotein-free serum, the nanoparticles have a lower IC50 (inhibitory concentration 50%) and a higher selective index in cancer cells as compared to Docetaxel in its regular formulation (free form), as demonstrated by the Examples of U.S. patent application Ser. No. 15/760,170.

In one aspect, the nanoparticles employed in the inventive methods may be spherical, with a size distribution range of about 20-150 nm. In some embodiments, the nanoparticles may include non-toxic surface active agents.

The surface active agents comprised in the nanoparticles preferably include Sodium Taurodeoxicholate and Poloxamer 188—both nontoxic agents—in contrast to other conventionally used surface active ingredients, such as Polysorbate 80. Toxicology of Intravenously administrated Poloxamer 188 indicates that its systemic toxicity is low. The intravenous LD50 was reported to be greater than 3 gm/Kg of body weight in both rats and mice. More recently, it has been described as one of the best pharmaceutical excipients for drug delivery; furthermore, it has been proven to have a neuroprotective effect once it passes through the BBB. (See Domb, Abraham J., Joseph Kost, and David Wiseman, Handbook of Biodegradable Polymers, (1998); Patel, H. R. et al. (2009); and Frim, D. M. et al. (2004)). On the other hand, Sodium Taurodeoxicholate is a naturally occurring surfactant (bile salt) and, thus, it is not expected to have undesirable or toxic side effects.

Regarding the ApoE ratio and concentration needed to have active drug delivery, Nelson et al. describes the use of 8-12% of apolipoprotein and a purification step, in order to eliminate all unbound proteins. According to embodiments of the invention, only 1% is needed to have the ApoE3 adsorb into the nanoparticles for targeted delivery. This also leads to fewer manufacturing steps to eliminate ApoE excess, thus making the manufacturing process more effective. In embodiments of the invention, the nanoparticles are loaded with a mass ratio of therapeutic agent to ApoE3 in the nanoparticle of from 1.1 to 3.3. A molar ratio of the therapeutic agent molecules per each recombinant ApoE3 molecule is preferably from 45 to 140.

An additional advantage of the lipid nanoparticles used in the methods of the invention includes the presence of the lipid core with a high retention capacity for liposoluble active ingredients without the need for conjugation. Although it has been mentioned in prior publications that no covalent modification of the active substance may be required for incorporation into a LDL particle, conjugation of active ingredients is common in order to keep the active ingredient inside the nanoparticle for a longer period of time, resulting in increased stability and avoidance of uptake of the active ingredient by non-targeted cells. Despite not being conjugated, in vitro tests showed that in human plasma the therapeutic agent is kept inside the lipid nanoparticles of the invention for at least 72 hours, and then transported by the nanoparticles without significant loss. Furthermore, when comparing a drug in free form to the nanoparticles used herein, after 72 hours, the nanoparticles showed lower release of the active ingredient when compared with the drug (TAXOTERE). As shown in U.S. patent application Ser. No. 15/760,170 (Example 4), with respect to TAXOTERE, the use of these nanoparticles for target delivery resulted in less toxic effects of the drug.

The stability of the lipid nanoparticles is yet another advantage over previously described LDL particles. Unlike Nelson's product, which is stable for only 2 weeks at 4° C., stability results for compositions of nanoparticles loaded with docetaxel according to embodiments of the invention have demonstrated that the liquid formulation is stable for at least 30 days at 4° C., without significant changes in the nanoparticle size, polydispersity, Z potential and active ingredient content (assay). Also, no increase of the active ingredient impurity levels has been detected. Furthermore, a lyophilized composition was found to be stable for at least 18 months at 25° C., without significant changes in particle size, polydispersity, Z potential and active ingredient content (assay). Also, the level of impurities for the active ingredient does not increase at higher rates than what it does in the reference products.

The lipid nanoparticles employed in methods of the invention not only structurally distinguish over previously described nanoparticles or similar artificial carriers, but also distinguish based on the unexpected properties resulting from the specific combination of components.

For example, McChesney et al. (U.S. Patent Application Publication No. 2015/0079189) describes synthetic low density lipoprotein (LDL) nanoparticles for the purpose of targeted cancer therapies These nanoparticles are comprised of a mixture including phospholipids, triglycerides, cholesterol ester, and free cholesterol, but are not coated with proteins triggering clearance processes in the tissues of the reticuloendothelial system, as previously mentioned. The nanoparticles of the invention, on the other hand, require the therapeutic agent to be dissolved in the triglyceride component (e.g., Castor Oil) in the nanoparticle core. Moreover, the lipid nanoparticles of the invention do not trigger an immunogenic response and thus allow for the use of ApoE in the formulation. As it has been shown that each individual has different levels of apolipoproteins in the body based on the varying physiological conditions of each individual, the amount of Apo proteins available results in a wide range of variability upon administration of the nanoparticles (see e.g., Liu et al., 2015). The presence of non-immunogenic ApoE3 in the nanoparticles used in the methods of the invention, however, overcomes this difficulty. As demonstrated by the Examples, the native ApoE3 does not bind or binds very poorly to the nanoparticle after intravenous injection, and the presence of ApoE3 in the nanoparticles selectively increased their targeting to cells. In this regard, the nanoparticle with ApoE3 reaches the target tissue 20% more efficiently than the nanoparticles With no attached apolipoprotein. (See Example 10 of U.S. patent application Ser. No. 151760,170).

As further demonstrated and explained in U.S. patent application Ser. No. 15/760,170 (Example 6), toxicity of the therapeutic agent is reduced when it is within the nanoparticle. Drug toxicity is even lower when facing a situation of active transport to targeted specific tissues, compared to encapsulated drug but without the Apo E3 to generate the active transportation.

Kreuter et al., in its publication titled “Apolipoprotein-Mediated Transport of Nanoparticle-Bound Drugs Across the Blood-Brain Barrier” describe a nanoparticle formulation which uses Polysorbate 80 for the attachment of ApoE. Their results suggest that the presence of Polysorbate 80 is needed in order to achieve the attachment of the ApoE to the nanoparticle. However, the inventors found and embodiments of the invention provide for the preparation of a stable nanoparticle formulation with ApoE bonded thereto without the need for Polysorbate 80. That is, the apolipoprotein component, or ApoE3, is bonded to the surface of the nanoparticle without Polysorbate 80. The toxic effects of tensoactives, such as Polysorbate 80, are well known, and the pharmacological and biological effects caused by tensoactives have been described as acute as hypersensitivity reactions, peripheral neuropathy, cumulative fluid retention syndrome, etc. That is the reason why efforts have been made to avoid the use of toxic surfactants and co-surfactants. (See Coors et al., 2005).

### IV. Administration and Delivery of Nanoparticles

In methods of the invention, ApoE-modified lipid nanoparticles loaded with a therapeutic agent are administered to a subject in need of treatment to effectively deliver the therapeutic agent to target cells or tissue. In some embodiments, improved delivery methods are provided, comprising administering to a subject an ApoE-modified lipid nanoparticle comprising a therapeutic agent so as to deliver the therapeutic agent across the blood-brain barrier to the desired or target cell or tissue.

It will be appreciated that that the effective amount of the lipid nanoparticles, as well as the route or mode of administration of the nanoparticles (and/or the therapeutic agent encapsulated in the nanoparticles) may vary according to the nature of the therapeutic agent to be administered or the condition to be treated. The specific dosage to be administered is of an amount deemed safe and therapeutically effective for the particular patient under the particular conditions and may be dependent on the mode of administration thereof.

The modes of administration may include any convenient route, including parenteral, enteral, mucosal, or topical. For example, administration according to the methods of the invention may be subcutaneous, intravenous, topical, intramuscular, intraperitoneal, transdermal, rectal, vaginal, intranasal, or intraocular.

In another embodiment, delivery of the therapeutic agent is by intranasal administration of the nanoparticles comprising the same, this mode being particularly useful in treatments of the brain and related organs (e.g., meninges and spinal cord). In another embodiment, the delivery of the therapeutic agent(s) is by intravenous administration of the same, which is especially advantageous when a longer-lasting intravenous formulation is desired.

Parenteral administration of a therapeutic agent according to methods of the invention includes modes of administration other than enteral and topical administration, usually by injection, including (without limitation) intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion.

In therapeutic applications, an effective amount of therapeutic agent-containing lipid nanoparticles can be administered to a subject by any mode allowing the nanoparticles to be taken up by capillary endothelial cells. That is, delivery of the therapeutic agents (drugs) to target cells and tissues preferably occurs by an active receptor-mediated process known as transcytosis. Notably, transcytosis occurs naturally in brain capillary endothelial cells, for example as a means of importing cholesterol and essential fatty acids into the brain.

Included within the scope of the invention are formulations comprising at least one ApoE-modified lipid nanoparticle as described herein for human or veterinary use, such as pharmaceutical compositions. Such compositions may further comprise pharmaceutically-acceptable carriers or excipients, optionally with supplementary medicinal agent. In embodiments, the pharmaceutically-acceptable excipient is selected from the group consisting of sucrose, sodium taurodeoxycholate, Poloxamer 188, sodium acid phosphate, potassium hydrogen phosphate, sodium chloride and potassium chloride. Conventional carriers, such as glucose, saline, and phosphate buffered saline, may also be used in such compositions.

In embodiments, the compositions may contain pharmaceutically acceptable excipients as required to approximate physiological conditions, such as pH adjusting and buffering agents, tonicity adjusting agents and the like. Other ingredients which may be included in the pharmaceutical compositions of the invention are known in the art and described in, e.g., Genaro, Remington's Pharmaceutical Sciences, Mack Publishing Co., Easton, Pa., (1985). Concentrations of the lipid nanoparticles in compositions within the scope of the invention can vary widely, such as from less than about 0.3% or at least about 1%, to as much as 5-10% by weight, depending on the type of composition, desired dosage and mode of administration.

In certain embodiments, the lipid nanoparticles may be formulated for controlled release, such that the release of the therapeutic agent from the nanoparticle is maintained to achieve the desired therapeutic level of the therapeutic agent in blood or tissue for an extended period (hours or days).

In still other embodiments, the invention provides a method of treatment that includes administering a therapeutically effective amount of a therapeutic agent enclosed in the lipid nanoparticles, whereby the lipid nanoparticles of the invention may include a targeting function due to the attachment of ApoE3. Targeting is a major advantage in, e.g., treatments of malignant tissues that have shown to have enhanced receptor expression, due to the favored uptake of a therapeutic agent encased in the nanoparticles. Furthermore, certain therapeutic agents, when encapsulated in the nanoparticles, may be used to target the necessary tissue (e.g., kill cancer cells or tumors more effectively) than the free drug, while reducing the impact the drug would otherwise have on normal tissues.

Methods for delivery of an agent to a discrete area of the brain are well known in the art, and can include the use of stereotactic imaging and delivery devices. The present invention encompasses any suitable method for intracranial administration of a targeted delivery composition to a selected target cell or tissue, including injection of an aqueous solution and implantation of a controlled release system.

### V. Treatment Methods

As discussed herein, the nanoparticles with ApoE used in methods of the invention bond to LDL receptors and have been found to be involved in transcytosis of LDL across the blood-brain barrier. Furthermore, when administered systemically, these nanoparticles have a differential uptake in brain tissue, as well as in lung, kidney and liver tissues. Therefore, targeted therapies described herein can lead to a reduction of undesirable side effects, toxic effects, as well as the dosage of administered chug, which further results in a general decrease in toxicity and cost.

Accordingly, provided herein are methods for: (i) delivering and/or improving targeted delivery of a therapeutic agent across the blood-brain barrier to a target cell or tissue in a subject; (ii) administering a therapeutically effective amount of ApoE-modified lipid nanoparticles loaded with a suitable therapeutic agent to a subject in need thereof for treatment of brain tissue disorders, infections, and related conditions; (iii) administering and delivering contrast agents for providing main diagnostic information; and (iv) administering a topical composition comprising a therapeutically effective amount of ApoE-modified lipid nanoparticles loaded with a suitable therapeutic agent to the skin of a subject suffering from certain skin conditions so as to achieve targeted delivery of the therapeutic agent without undesirable side effects resulting from the therapeutic agent being in contact with the skin.

A. Brain Disorders

With respect to a first class of embodiments, the invention provides for methods of treating brain diseases and related conditions, comprising administering to a subject an effective amount of ApoE-modified lipid nanoparticles that contain a suitable therapeutic agent loaded therein. Pursuant to treatment methods of the invention, the uptake of ApoE3-modified nanoparticles is significantly higher than uptake resulting from administration of the same, but non-targeted particles.

Brain tissue disorders include, but are not limited to, neurological disorders, neurodegenerative diseases, cerebrovascular ischemia, traumatic brain injury, stroke, small-vessel cerebrovascular disease, brain tumors, epilepsy, migraine, narcolepsy, insomnia, chronic fatigue syndrome, mountain sickness, encephalitis, meningitis, and AIDS-related dementia.

In other embodiments, the methods of the invention are used to treat central nervous system disorders where the central nervous system disorder is a tumor or cancer. Brain tumors include any intracranial tumor created by abnormal and uncontrolled cell division, normally either found in the brain itself, the lymphatic tissue or blood vessels, in the cranial nerves, in the brain envelopes (meninges), skull, pituitary and pineal gland, or spread from cancers primarily located in their organs (metastatic tumors). Primary brain tumors are commonly located in the posterior cranial fossa in children and in the anterior two-thirds of the cerebral hemispheres in adults, although they can affect any part of the brain. Most primary brain tumors originate from glia (gliomas), astrocytes, oligodendrocytes, or ependymal cells.

Other varieties of the primary brain tumors include primitive neuroectodermal tumors, tumors of the pineal parenchyma, ependymal cell tumors, choroid plexus tumors, neuroepithelial tumors of uncertain origin. A type of primary intracranial tumor is primary cerebral lymphoma, also known as primary central nervous system lymphoma, which is a type of non-Hodgin's lymphoma. The term “glioma” refers to a tumor originating in the neuroglia of the brain and spinal cord. Gliomas are derived from the glial cell types, such as astrocytes and oligodendrocytes, thus gliomas include astrocytomas and oligodendrogliomas, as well as anaplatic gliomas, glioblastomas, and ependymomas, astrocytomas and ependymomas can occur in all areas of the brain and spinal cord in both children and adults.

B. Infections

In some embodiments, the methods disclosed herein are useful for treating pathogen infections, preferably infections of brain tissue and also systemic infections, including (but not limited to) infections of the tissues of, or covering, the brain and spinal cord, including (but not limited to) infections caused by Meningococci (meningitis). In embodiments, such treatment methods include administering a suitable therapeutic agent loaded onto an ApoE-modified lipid nanoparticle as described herein to a subject in need thereof.

Infections of brain tissue, in particular, may include fungal infections. In specific embodiments of the invention, treatment of fungal infections comprises administering a therapeutically effective amount of Amphotericin B in an ApoE-modified lipid nanoparticle as described herein.

From commercial formulations of Amphotericin B FUNGIZONE micelle with sodium desoxycholate has higher affinity for sticking to plasma HDL (75% of the total) than to LDL, whereas this percentage rises to an average of 90% for AMBISONE. This suggests that the lower distribution of AmB in LDL when negatively charged liposomes are used may explain in part the lower toxicity associated with this intravenous administration. (See Kishor M. Wasan et al., “Roles of Liposome Composition and Temperature in Distribution of Amphotericin B in Serum Lipoproteins,” Antimicrobial Agents and Chemotherapy, Vol. 37, No. 2, pp. 246-50 (1993)). On the other hand, it has been reported that the cause of in vivo toxicity of Amphotericin B is the formation of blood complexes with low density lipoproteins (LDL) and very low density lipoproteins (VLDL), and that preventing their formation reduces their toxicity (Barwicz J. et al., “Inhibition of the interaction between lipoproteins and amphotericin B by some delivery systems,” Biochem. Biophys. Res. Commun. 181(2): 722-8 (1991)).

It has also been suggested that renal toxicity of Amphotericin B is proportional to the plasma concentration of LDL (Kishor M. Wasan et al., “Influence of Lipoproteins on Renal Cytotoxicity and Antifungal Activity of Amphotericin B.,” Antimicrobial Agents and Chemotherapy. Vol. 38, pp. 223-27 (1994)). In turn, it has been shown that hypercholesterolemia in mice with deficiency of low density lipoprotein receptors (LDL-R) increases the susceptibility of these animals to systemic candidiasis (Netea M. G. et al., “Hyperlipoproteinemia enhances susceptibility to acute disseminated Candida albicans infection in low-density-lipoprotein-receptor-deficient mice,” infect. Immun. 65:2663-7 (1997)).

Existing data appears to suggest that a lipid-rich environment promotes greater growth of C. albicans. For example, it has been shown that hyperlipoprotein LDLR −/− mice are more susceptible to disseminated candidiasis due to increased fungal growth in their organs. (Netea M. G. et al., “Hyperlipoproteinemia enhances susceptibility to acute disseminated Candida albicans infection in low-density-lipoprotein-receptor-deficient mice,” infect. Immun. 65: 2663-7 (1997)). Although lipid profiles differ between mice and humans, the results of both studies suggest that hyperlipidemia may have detrimental effects by stimulating the growth of C. albicans in both species.

Candidiasis can affect the central nervous system and induce encephalopathy and microabscesses (Sánchez-Portocarrero, J. et al., “The central nervous system and infection by Candida species,” Diagn. Microbiol. Infect. Dis. 37,169-179 (2000); Kang, C.et al., Anidulafungin treatment of candidal central nervous system infection in a murine model,” Antimicrob. Agents Chemother. 53: 3576-78 (2009)). Candida meningo encephalitis has a high morbidity and mortality in immunocompromised individuals such as patients with AIDS or in situations of prolonged immunosuppression, for example hematological malignancies and transplants (Sánchez-Portocarrero, J. et al., (2000)). In premature children and pediatric patients, meningoencephalitis caused by Candida is a particularly serious nosocomial fungal infection (Groll, A. H. et al., “Comparative efficacy and distribution of lipid formulations of amphotericin B in experimental Candida albicans infection of the central nervous system,” J. Infect. Dis. 182: 274-82 (2000); Strenger, V. et al., “Amphotericin B transfer to CSF following intravenous administration of liposomal amphotericin,” B. J. Antimicrob. Chemother,” 69:2522-26 (2014)).

Therefore, Amphotericin B (AmB), a hydrophobic antibiotic with a broad antifungal spectrum, is commonly used in the treatment of severe systemic fungal infections (Strenger, V. et al., “Amphotericin B transfer to CSF following intravenous administration of liposomal amphotericin,” B. J. Antimicrob. Chemother. 69: 2522-26 (2014)). However, the blood-brain barrier remains a pharmacological barrier to existing commercial formulations of Amphotericin B (Groll, et al., (2000); Shao, K. et al., Angiopep-2 modified PE-PEG based polymeric micelles for amphotericin B delivery targeted to the brain,” J. Control. Release 147,118-26 (2010)).

The deoxycholate of Amphotericin B (FUNGIZONE) and liposomal AmB (AMBISOME) have shown good distribution and access to the central nervous system (CNS) in animal models and led to the complete eradication of Candida albicans from the brain. (See Groll, A. H. et al., “Comparative Efficacy and Distribution of Lipid Formulations of Amphotericin B in Experimental Candida albicans. Infection of the Central Nervous System” (2000); Clemons, K. V. et al., “Comparative efficacies of conventional amphotericin B, liposomal amphotericin B (AmBisome), caspofungin, micafungin, and voriconazole alone and in combination against experimental murine central nervous system aspergillosis,” Antimicrob. Agents Chemother. 49,4867-75 (2000); Shao, K. et al., “Angiopep-2 modified PE-PEG based polymeric micelles for amphotericin B delivery targeted to the brain,” J. Control. Release 147: 118-26 (2010)).

According to methods of the invention, the ApoE-modified lipid nanoparticles loaded with Amphotericin B formulation are administered to a subject in need thereof to treat intracerebral infections of Candida albicans, resulting in enhanced delivery of the therapeutic agent (Amphotericin B) to the brain than the therapeutic agent in its conventional or free form. The formulation administered in the methods of the invention—ApoE-modified lipid nanoparticle comprising Amphotericin B formulation as a therapeutic agent—avoid the variability in treatment of a subject with an increased concentration of LDL, HDL, VLDL, and also reduce the cellular toxicity of the low hemolytic potential.

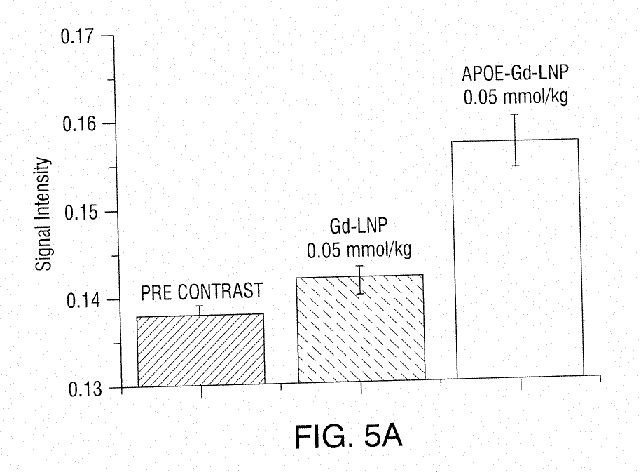

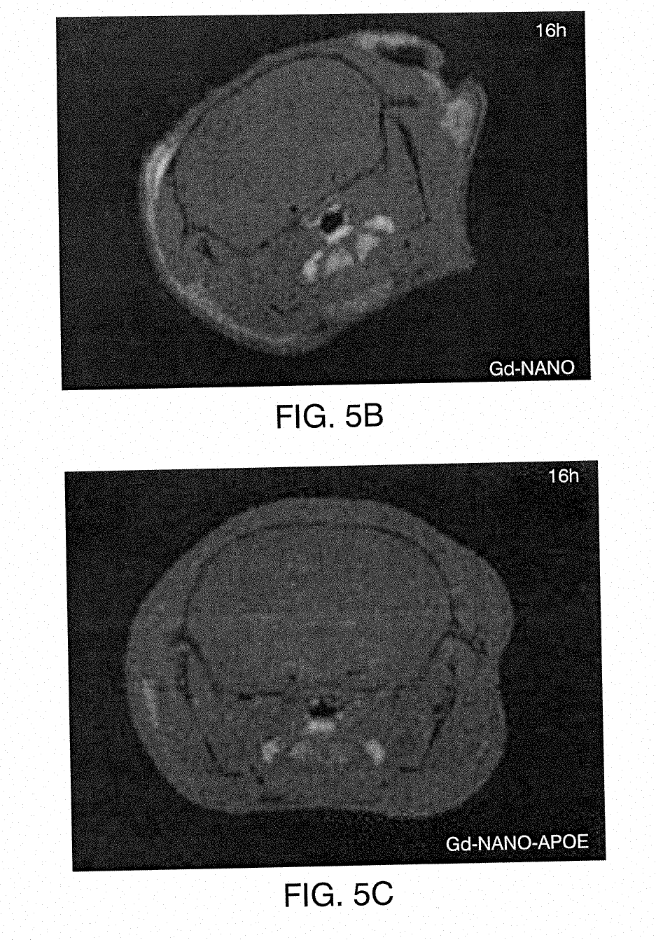

As shown in FIG. 5, amphotericin B can be loaded and in stable in lipid nanoparticles with ApoE as used herein. As further demonstrated, when injected in a murine model, the nanoparticle with therapeutic agent (Amphotericin B) reaches liver, brain, lung, and kidney tissue. Furthermore, in vitro results show that Amphotericin B loaded into the lipid nanoparticles with ApoE possess MIC of half of that of AMBISOME and has a substantially lower hemolytic power than FUGIZONE at a similar concentration. Unifying the concentration of Amphotericin B in all patients through the use of an Amphotericin B formulation loaded onto the ApoE-modified lipid nanoparticles according to embodiments of the invention, due to enhanced delivery of the therapeutic agent to target brain tissues, would provide a suitable and improved treatment of Candida albicans cerebral infections delivery of the therapeutic agent to target tissues in the brain better arrival to the brain to treat intracerebral infections of Candida albicans.

C. Diagnostic Methods

As indicated herein, it is furthermore an object of the present invention to improve MRI specificity using cell markers and the properties of paramagnetic and superparamagnetic particles, which can be utilized to be detected with MRI in small quantities, e.g., Magnetite and Gadolinium.

MRI is the mechanism by which images of super anatomical resolution (0.1×0.1 mm) can be obtained, and functions of soft tissues in vivo simultaneously mapped. Gd-based contrast agents are commercially available. However, accumulation of these agents is solely based on differences in the vasculature between abnormal and normal tissues. Thus, MRI recognition of specific tumor types, for example, is not achieved. That is, traditional MRI pulse sequences depict regional differences in tissue composition, and the use of various iron-based MRI contrast agents that have also been developed has shown to result in signal loss. In addition, there are several commercially available MRI contrast agents that use Magnetite with an oleic acid coating. However, this formulation does not allow redirecting the magnetic nanoparticles a specific targeted cell or tissue.

Accordingly, in certain embodiments, a therapeutic agent to be administered by the methods of the invention can be a chemical entity or biological product, or combination of chemical entities or biological products, administered to a subject for imaging purposes in the subject. Specifically, the therapeutic agent(s) administered according to methods of the invention can be selected from “imaging agents” “contrast agents.” Included within the scope of the invention are diagnostic agents, such as specific contract media for brain imaging, that are currently not used because of poor penetration into the brain upon systemic administration of the diagnostic agent in its free form or using known delivery methods.

Although penetration and tomographic imaging potential are limited, near infrared (NIR) optical imaging does offer unique advantages over radioactive imaging modalities for noninvasive detection of subsurface tumors. It is safe and inexpensive and permits differentiation of tumors and normal tissues based on differences in tissue absorption or fluorescence. These tissues are relatively transparent to the NIR light. Target-specific NIR probes can overcome the small intrinsic contrast between tumors and normal tissues, thereby providing high sensitivity and specificity in tumor detection.

In embodiments of the invention, cells can be labeled with lipophilic carbocyanine dyes (e.g., DiI, DiO, DiA, DiR and derivatives). Other fluorescent contrast agents for clinical applications are indocyanine green, methylene blue and fluorescein. Carbocyanine dyes have long wavelength absorption, high extinction coefficients (>100,000), and high fluorescence quantum yields, which are the ideal properties of NIR probes.

Therefore, according to further embodiments of the invention, administering/delivering contrast agents, such as magnetites, within the ApoE-modified lipid nanoparticle to target cells tissues will reduce the non-desired effects and allow for better resolution in the MRI. Moreover, the lipid nanoparticles use active transport directed by ApoE3, and have higher uptake in cancer cells than in healthy tissues. Thus, administration of the ApoE-modified lipid nanoparticles loaded with a contrast agent could also be used to identify the presence of abnormal tissues.

In some embodiments, administration of a diagnostic/imaging agent may be included within or combined with the treatment methods described herein. Administering nanoparticles loaded with an imaging agent, such as, e.g., gadolinium, can be performed alone or in conjunction with administration of a treatment agent (e.g., chemotherapeutic agent).

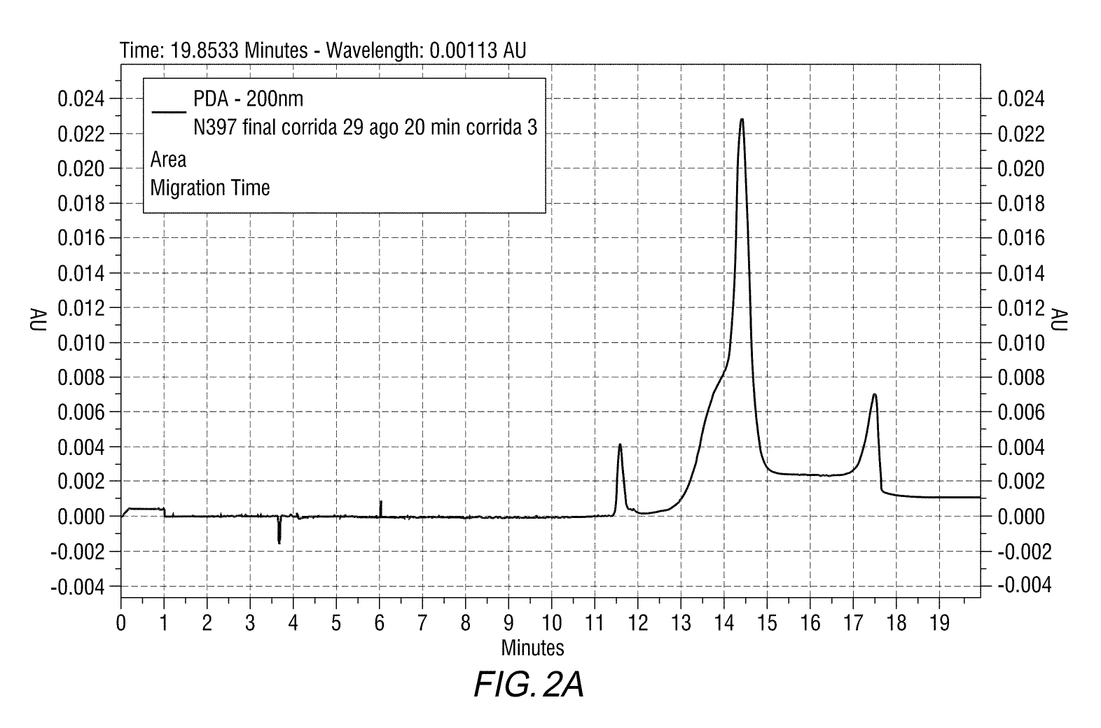

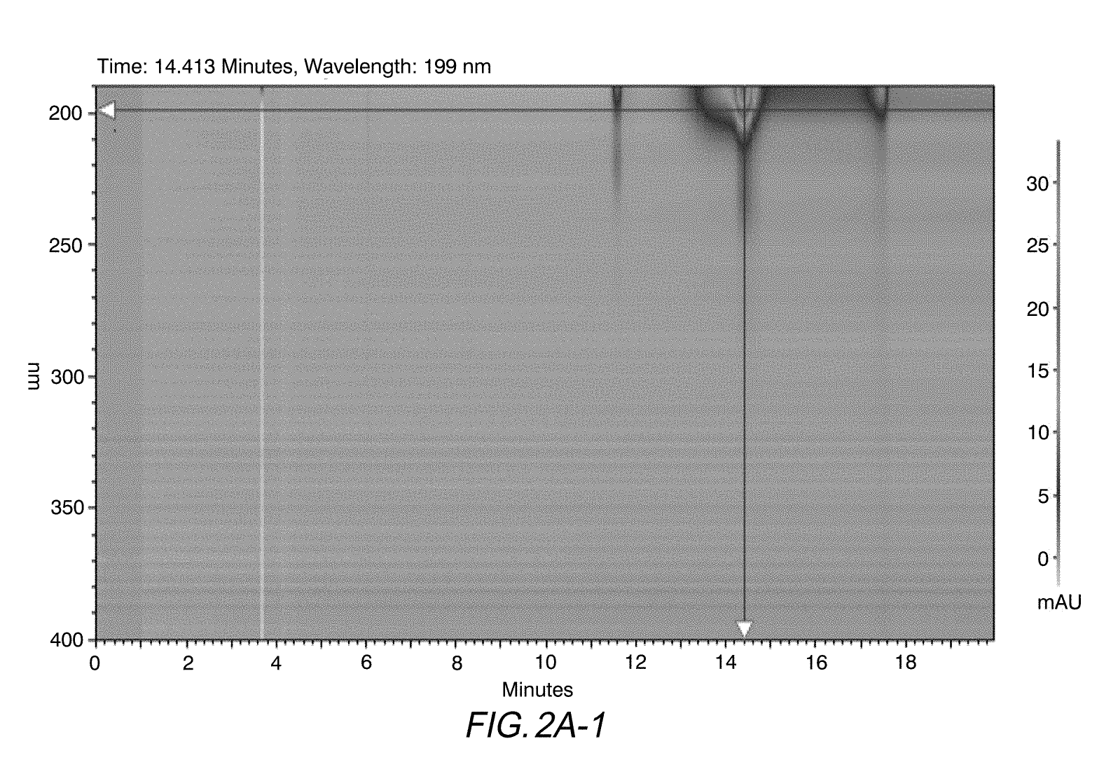

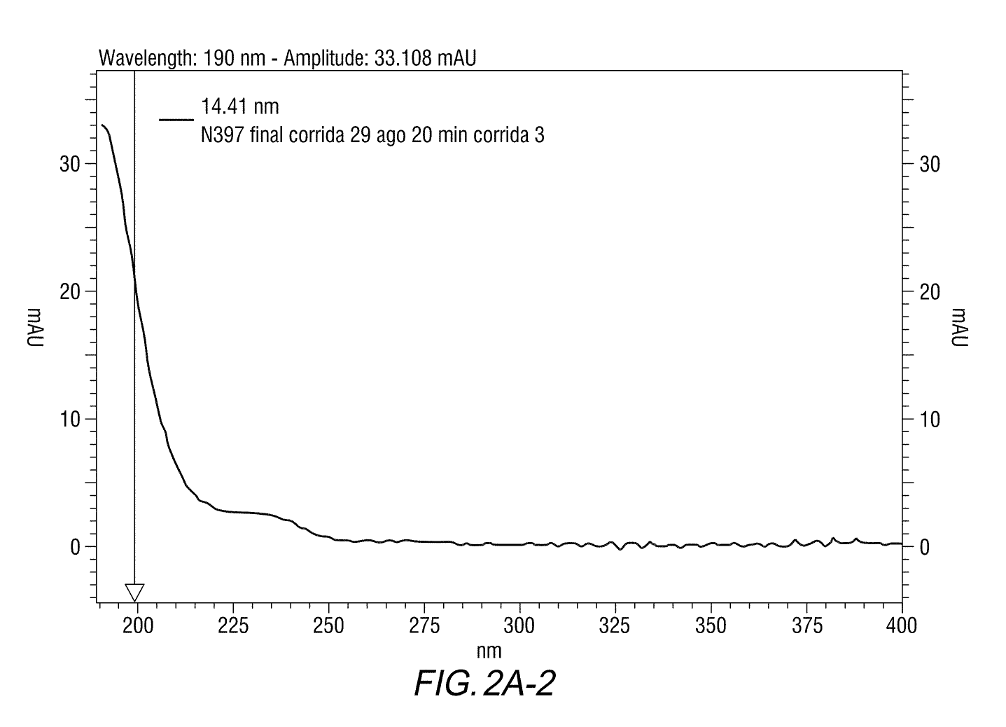

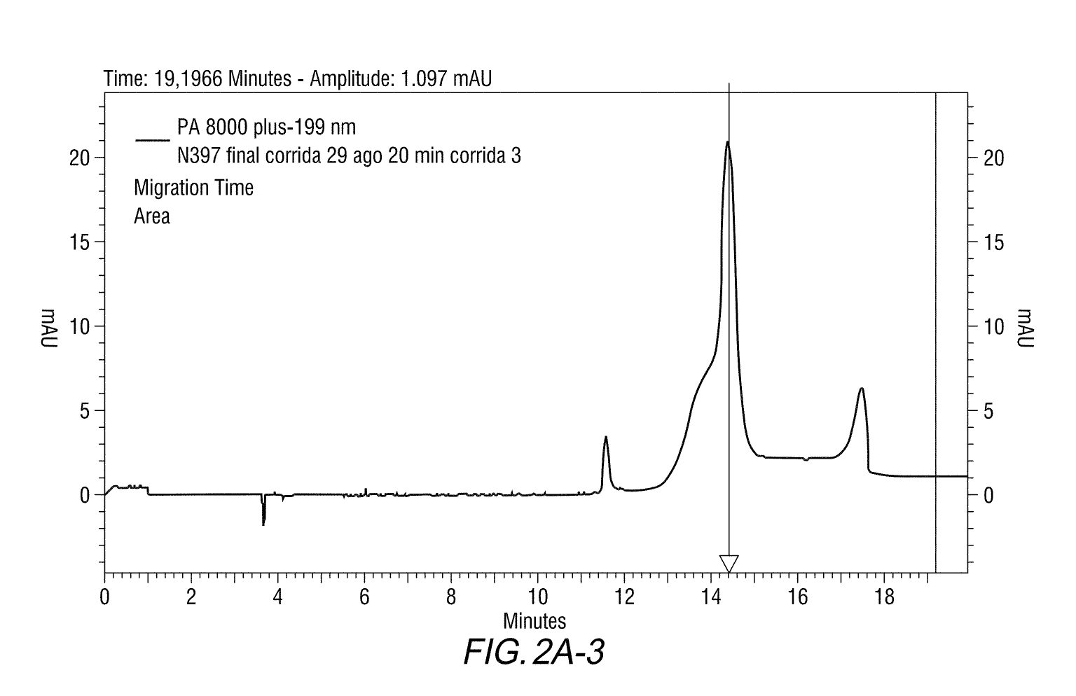

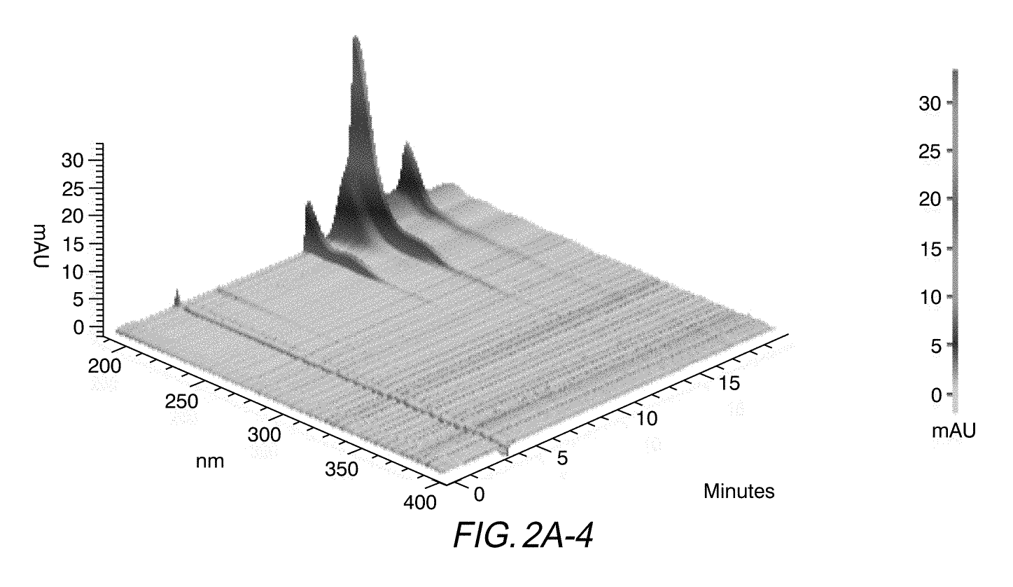

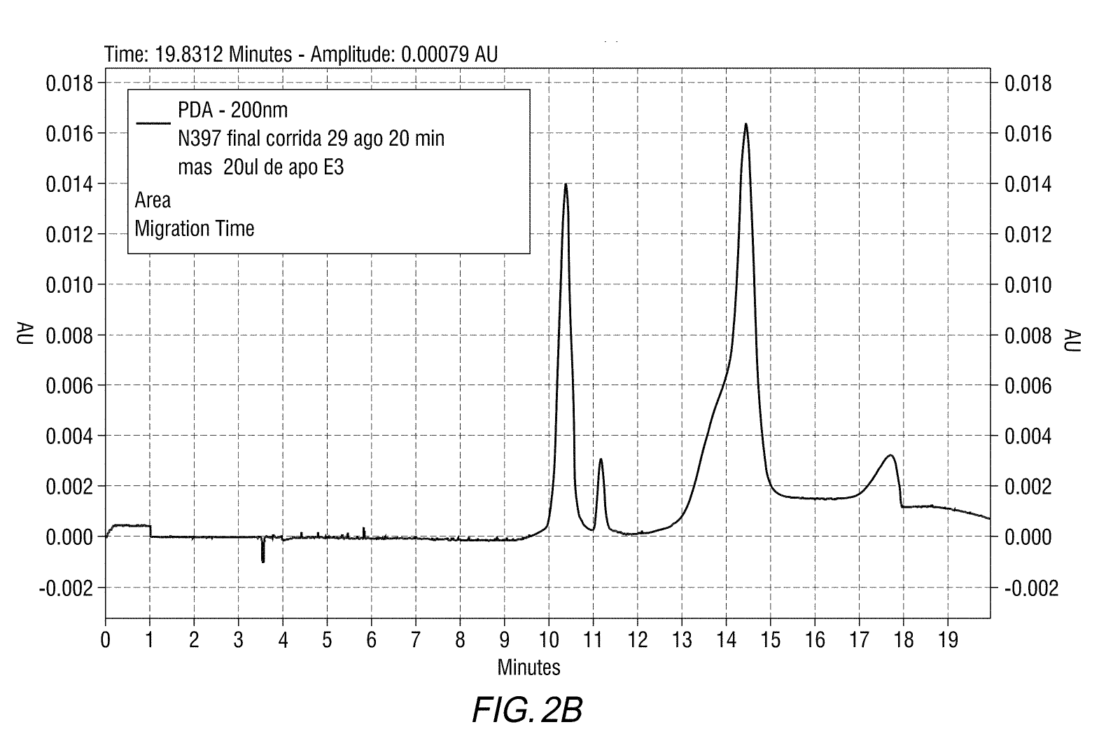

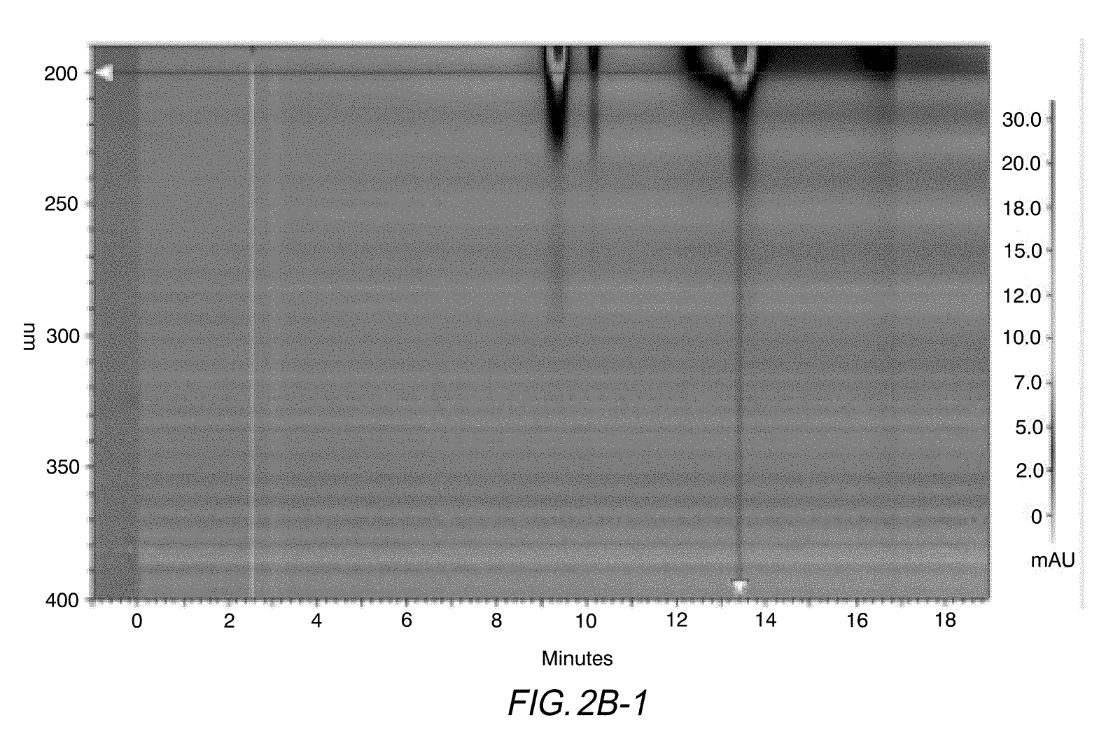

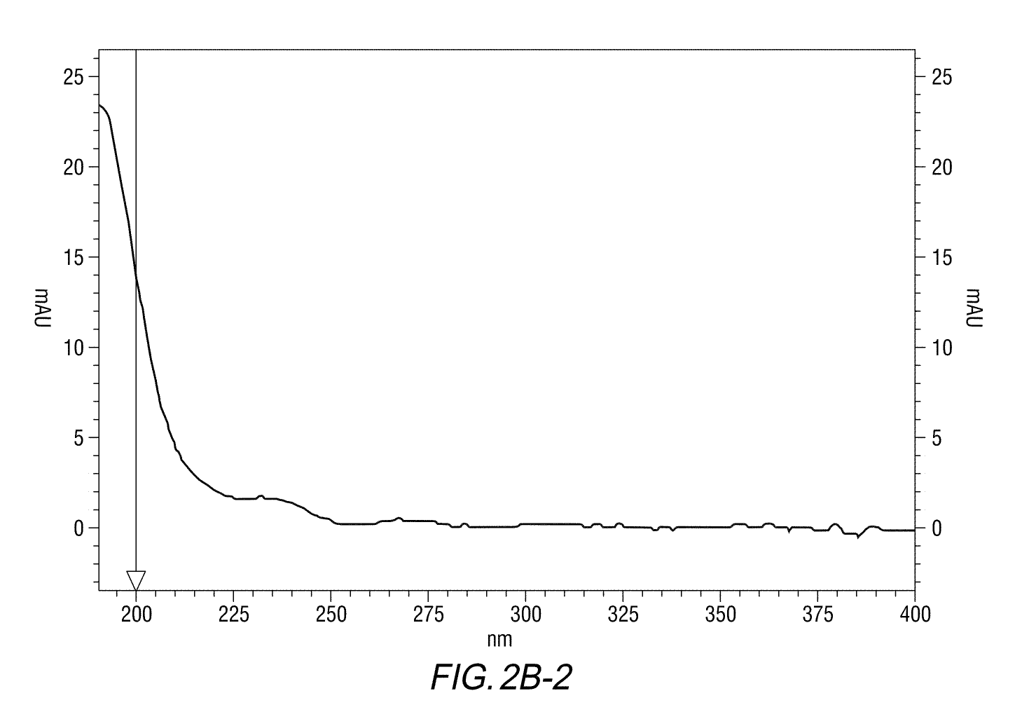

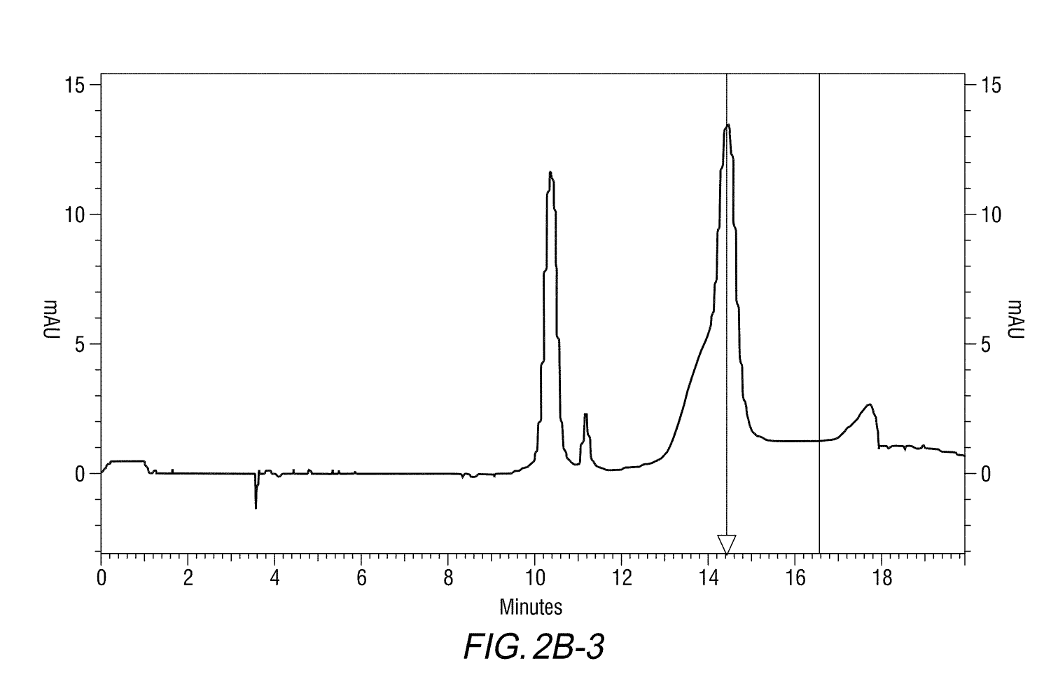

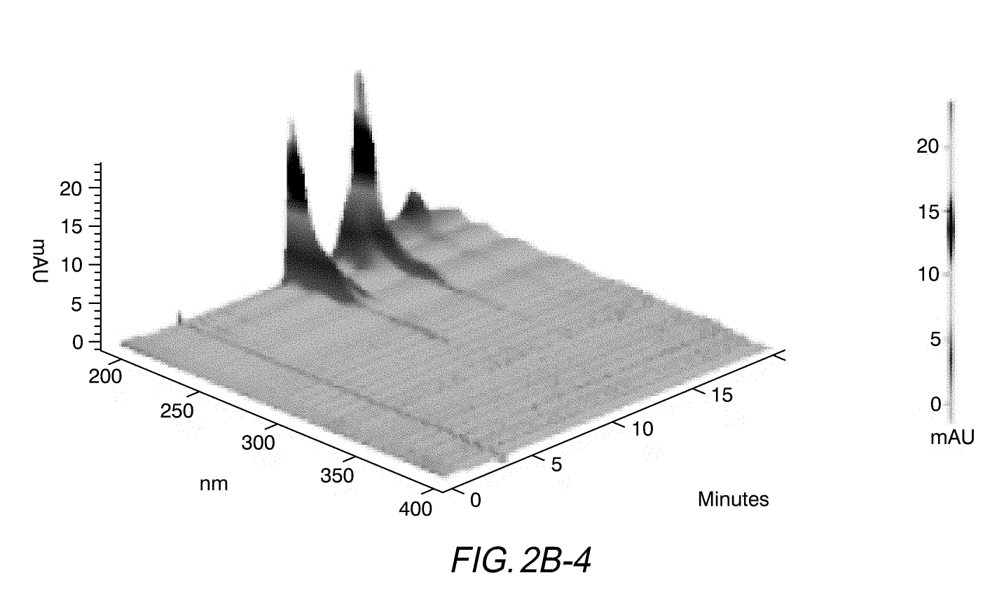

As demonstrated by Example 9 below, Magnetite of 9 nm can be loaded in the stable ApoE-modified lipid nanoparticles described herein. FIG. 2 shows that the uptake of ApoE-modified lipid nanoparticles with Gadolinium is significantly higher than the non-targeted (without ApoE) particles in tumor cells, suggesting that the ApoE3 targeted nanoparticles enter the cells via the LDLr. Moreover, the ApoE-modified particles with Gadolinium, upon injection, have the ability to cross the blood-brain barrier. Furthermore, in vivo results show that the lipid nanoparticles with DIR and ApoE are preferentially captured in cells/tissues of liver, lung, kidney and brain.

Therefore, also encompassed within the scope of the invention are methods of delivering ApoE-modified lipid nanoparticles loaded with imaging agent(s) to target cells/tissues for monitoring of tissues or tumors that overexpress LDL receptors.

D. Skin Conditions

As a further object of the invention, provided are methods for treatment of skin conditions, primarily those associated with reduced collagen production. It is well know that topical tretinoin (retinoic acid) improves fine wrinkles associated with damage caused by exposure to sunlight (photodamage) and it is also believed that the reduction of collagen levels in areas of the skin exposed to the sun is an etiological component. Mice exposed to ultraviolet radiation acquire fine wrinkles similar to those seen in humans with photo damage. When such mice are treated with topical tretinoin, the erasure of the wrinkles occurs in association with the appearance of a sub-epidermal repair area detectable by routine light microscopy. (See Kligman A. M. et al., “Topical tretinoin for photoaged skin,” J. Am. Acad. Dermatol. 15:836-59 (1986); Weiss J. S. et al., “Topical tretinoin improves photoaged skin: a double-blind, vehicle-controlled study,” JAMA, 259: 527-32 (1998); Lever L et al., “Topical retinoic acid for treatment of solar damage,” Br. J. Dermatol. 122: 91-8 (1990); Weinstein G D, Nigra T P, Pochi P. E. et al., “Topical tretinoin for treatment of photodamaged skin: a multicenter study,” Arch Dermatol. 127: 659-65 (1991); Olsen E. A. et al. “Tretinoin emollient cream: a new therapy for photodamaged skin,” J. Am. Acad. Dermatol. 26: 215-24 (1992); Bissell D. L. et al. “An animal model of solar-aged skin: histological, physical, and visible changes in UV-inadiated hairless mouse skin,” SNAD Photochem. Photobiol. 46: 367-78, (1987)).

The finding of increased collagen I formation in photodamaged human skin treated with tretinoin suggests that tretinoin promotes clinical improvement by repairing dermal collagen. Furthermore, tretinoin is known to influence several cellular processes, such as cell growth and differentiation, cell surface alteration and its immune modulation. Many of their effects on tissues are mediated by their interaction with specific cellular and nucleic acid receptors. Cellular or cytoplasmic receptors include cellular retinoic acid binding protein (CRABP) types I and II and cellular retinol binding protein. (See Astrom A. et al., “Molecular cloning of two human cellular retinoic acid-binding proteins (CRABP),” J. Biol. Chem. 266: 17662-6 (1991)).

Topical treatment of acne vulgaris and dermatoheliosis (photodamage) was begun with RETIN-A (topical tretinoin) gel or cream, which stimulates the production of new non-adherent corneal cells within the follicular canal, accelerating the detachment of old cells from the superficial layers up to 6 times the normal rate of velocity.

Retin-A micro gel beads, loaded with tretinoin at 0.1%, 0.08% and 0.04%, is a new product that is superior to the traditional RETIN-A in gel or cream as a result of not exposing the skin to a high concentration of tretinoin, and reducing the side effects of erythema, peeling, itching and burning. This is due to the gradual release of tretinoin by the Retin-A micro gel beads that prevents a high concentration of the active substance.

In one embodiment of the invention, the ApoE-modified nanoparticle loaded with tretinoin avoids contact/interact between the tretinoin and a surface of the skin. Encapsulating tretinoin in the lipid nanoparticles reduces the sign effects compared with the referenced microspheres of Retin-A Micro gel. Furthermore, the described nanoparticles having a small size (Z average of 53 nm and PDI 0.1) are suitable for diffusion across the epidermis to reach the fibroblasts in the dermis. Pursuant to embodiments of the invention, the tretinoin is released intracellularly via endocytosis mediated by LDL-R, and this stimulates its nuclear receptor to further stimulate the production of pro-collagen and accelerate its metabolism.

In some embodiments, the invention relates to non-hyperkeratotic and non-hypertrophic actinic keratosis in adults. In such embodiments, the ApoE-modified lipid nanoparticle is loaded with Ingenol for targeted delivery thereof for the treatment of actinic keratosis. Ingenol is a molecule that binds and activates protein kinase C and, in biological systems, induces similar responses to phorbol esters. The concentration values of Ingenol are typically between 30 uM and 1 mM for biological activity. (See Clare M. Hasler et al., “Specific Binding to Protein Kinase C by Ingenol and Its Induction of Biological Responses,” Cancer Research, 52: 202-208 (1992)).

### VI. Disclaimer

While the invention has been described with respect to particular embodiments, it will be apparent to those skilled in the art that various changes and modifications may be made without departing from the spirit and scope of the invention defined in the appended claims. Such modifications are also intended to fall within the scope of the claims. Persons skilled in the art would recognize that there exist a broad range of possible clinical applications of the inventive methods described herein.

Embodiments of the invention are further illustrated by the following examples, which should not be construed as limiting.

### EXAMPLES

The following Examples serve to further illustrate specific embodiments and are not to be construed as limiting the scope of the invention in any way.

Preparation methods of the ApoE-modified lipid nanoparticles employed in the methods of the invention are those described in U.S. patent application Ser. No. 15/760,170, and as referenced in the Examples below.

### Example 1

### Stability Evaluation of

### Lipid Nanoparticles with Gadolinium

Particle stability was measured for: (a) lipid nanoparticles loaded with ApoE3 and charged with Gadolinium (Gd)—DOTAMA; and (b) nanoparticles without ApoE3 and charged with Gd. The particle stability was measured at 37° C. by measuring the relaxation rates of the nanoparticles in an isotonic NaCl/Hepes buffer for 48 hours under dialysis.

Results of the stability test showed that both nanoparticle formulations (a) and (b) are stable at the provided conditions for at least 48 hours, and that Gd remains inside the nanoparticles after reconstitution of the lyophilized nanoparticle.

### Example 2

### Selective Uptake of ApoE3

### Lipid Nanoparticles Charged with Gd (ApoE3-Np)

Human lung carcinoma cells (A549) and neuroblastoma cells (Neuro2a) were selected due to having an up-regulated low density lipoprotein receptor (LDLr) that specifically recognizes and bonds to apoproteins. Furthermore, the neurite extension in neural development is further enhanced in Neuro2a cells by entry of ApoE3 in a lipid environment.

The selective uptake of ApoE3-Np by these cells was evaluated. The cells were cultured in: (a) a lipoprotein-free serum with ApoE3-Np added; and (b) a lipoprotein-free serum with Np added. Both cultures were labeled with Gd-DOTAMA during 6 and 25 hours, and at a final Gd concentration of 25 μM. The nanoparticle uptake results are provided in FIG. 2, expressed as moles of Gd normalized to the mg of cell proteins (directly proportional to the cell number).

As shown in FIG. 2, the uptake of ApoE3-Np is significantly higher than the non-targeted particles in both types of tumor cells. That is, there is a differential uptake between the nanoparticles loaded with ApoE3 (ApoE3-Np) and those without ApoE3 (Np), suggesting that the ApoE3 targeted nanoparticles enter the cells via the LDLr. Additionally, ApoE3-Np/Np uptake ratio by the tumor cells increased as the incubation time increased, indicating that a longer incubation time enhances specific uptake of the ApoE3 targeted nanoparticles.

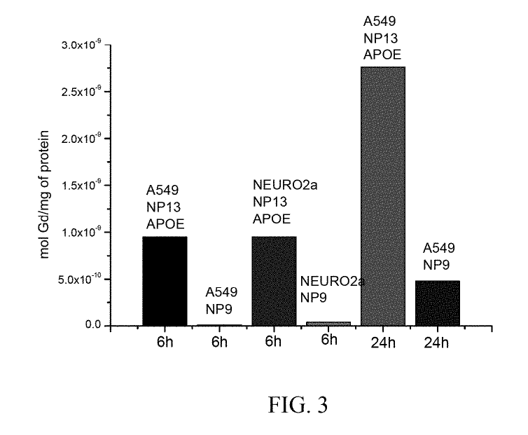

The in vitro cellular uptake demonstrated that conjugation with ApoE3 selectively increases targeting to cells, thus making them useftil for treatment or diagnostic methods. As confirmed by MRI images of cells incubated with ApoE3-Np and with Np and placed at the bottom of glass capillaries after washing, only cells incubated with ApoE3-Np appeared hyper intense with respect to the control (see FIG. 3).

### Example 3

### Biological Activity of ApoE3

### Lipid Nanoparticles Charged with Gd (ApoE3-Np)

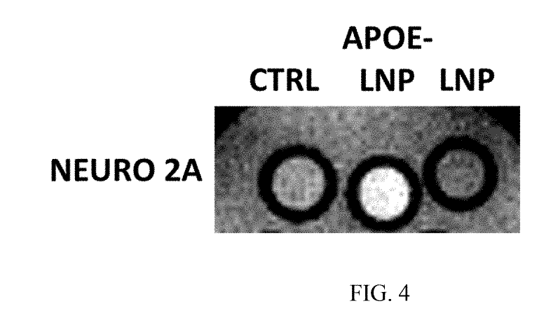

MRI tests were carried out to assess the capability of ApoE3-Np to cross the BBB in 8-week old male BALB/c mice. FIGS. 4A and 4B show results based on T1-weighted brain images, wherein red grey-scale pixels are those showing a SI increase by >3 SD of the pre-contrast brain image. These enhanced pixels represent about 8% of the total brain pixels in mice treated with the Gadolinium nanoparticle with ApoE3, whereas in mice treated with Gadolinium lipid nanoparticles without ApoE3 they represent only 0.4%. As shown in FIG. 4A, the measured signal intensity of the total cerebral tissue after injection of ApoE-Np was significantly higher than that measured after the injection of the same quantity of non-targeted nanoparticles (Np). These preliminary tests confirm that ApoE-Np have good tolerability and the ability to cross the blood-brain barrier.

### Example 4

### Nanoparticles Loaded with Amphotericin B

Organic Phase Preparation: 200 g of anhydrous ethanol, 1.03 g of egg yolk PL (Egg PC 80 lipoid), 1.58 grams of Castor oil USP, 0.09 grams of Cholesteryl oleate and 0.12 grams of cholesterol were added into a 250 ml round-bottomed flask inside a thermostatized bath with bubbling nitrogen previously heated to 40° C.; to this mixture an acid solution containing 0.11 g Amphotericin B USP; 0.103 grams of 1,2-Dimyristoyl-sn-glycero-3-phosphorylcholine (14:0 PC (DMPC) Lipoid GmbH, Germany), 0.046 grams of 1,2-Dimyristoyl-sn-glycero-3-phosphoglycerol (14:14 PG (DMPG) Lipoid GmbH, Germany) in 1 ml of mixture dichloromethane:methanol (1:1) with 25 ul of hydrochloric acid 2N. The final mixture was stirred until complete dissolution of all components.

Aqueous Phase Preparation: 800 grains of WFI (previously filtered with a 0.45 μm PVDF membrane), 0.4 grams of poloxamer 188 (Lutrol F68, BASF, Germany) and 0.2 grams of sodium taurodeoxycholate (New Zealand Pharmaceuticals LTD, New Zealand) were added to a 2 L glass Schott bottle inside a thermostatized bath with bubbling nitrogen previously heated to 40° C. The mixture was stirred with a 60 mm stirring bar at 500 rpm.

Nanoparticle Manufacture: To obtain the lipid nanoparticles, organic phase was injected into the aqueous phase (heated at 40° C. and stirred at 500 rpm) at a rate of 1-1.5 ml/sec using a 4-hole nozzle. The mixture was stirred at 250 rpm for 45 minutes. Then, the nanoparticles were concentrated by distillation under reduced pressure until the desired fat percentage value was reached (approximately 25 mg/ml of total lipids). After concentrating the nanoparticles, the solution was brought to pH 7.4 by adding a phosphate buffer solution

Recombinant ApoE3 Bonding to Nanoparticles: A 2 mg/ml ApoE3 solution (in phosphate buffer) was added to a 250 ml round bottom flask containing the produced nanoparticle solution with Amphotericin B until reaching a final concentration of 0.2 mg/ml ApoE3 in the solution. The resulting solution was then incubated at 40° C.±2° C. with orbital agitation for 60 minutes. The size (Z-average) and dispersion (PDI) of the resulting nanoparticles was then measured by DLS as shown in the Tables below.

### Example 5

### Nanoparticles with Amphotericin B

The size and PDI of the ApoE-modified lipid nanoparticles loaded with Amphotericin B was determined, while using ratios and proportions as per the bibliographic suggestions. For both types of lipid nanoparticles provided, nanoparticles the size and PDI was determined using dynamic light scattering (DLS). The composition of each composition and the size and Pdi results are show in Tables 4 and 5 below.

TABLE 4

| | | | Cholesteryl | | | Particle | |
|---|---|---|---|---|---|---|---|
| | Phospholipids | Castor Oil | Oleate | Cholesterol | mB | Size | |
| | (%) | (%) | (%) | (%) | (%) | (diameter) | D1 |
| 416 | 3 | 2 | 9 | 14 | 2 | 7.56 nm | 0.244 |
| 436 | 8 | 1 | 3 | 4 | 4 | 2.17 nm | 0.119 |

TABLE 5

| | | |
|---|---|---|
| 416 | DMPG:AmB | 54:6:3:2 |
| 436 | DMPG:AmB | 33:3:2:4 |

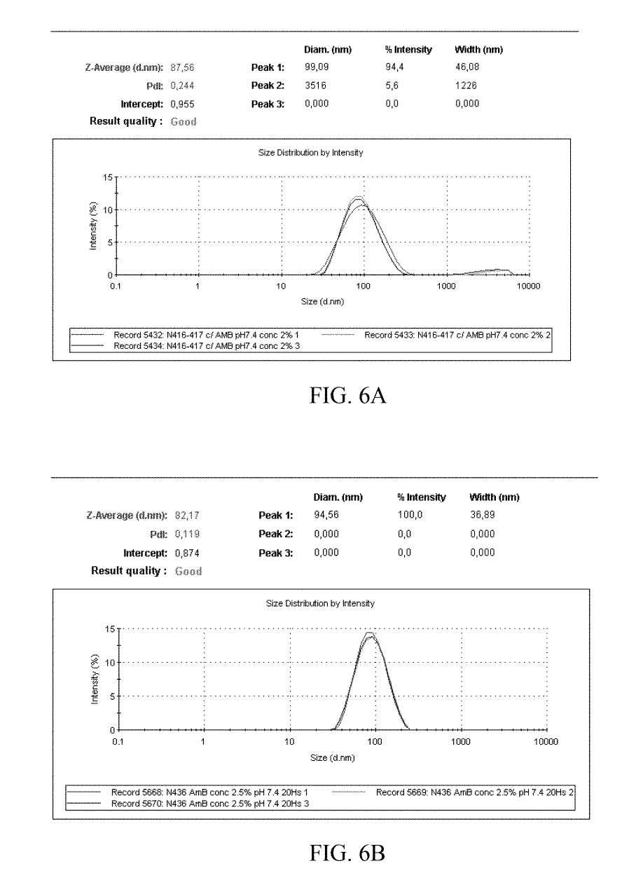

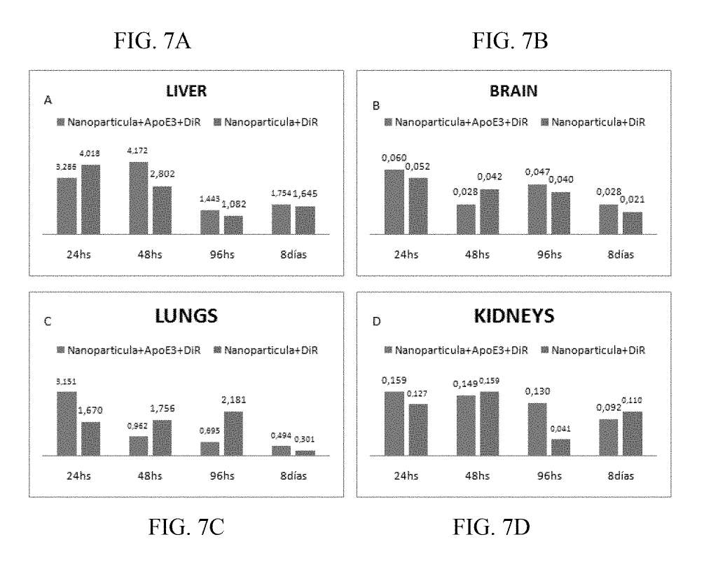

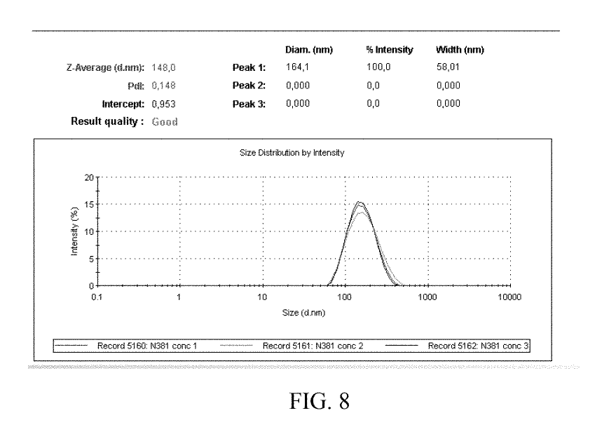

DLS results provided in FIG. 6A show the volume distribution of lipid nanoparticles for formulation N416 and FIG. 6B for N436. As shown in the Figures, Amphotericin B nanoparticles within the scope of the invention (N436) have an average size of 87.56 nm, and Pdi of 0.119 (below 0.2). On the contrary for the formulation as per the bibliographic information composition (N416), it is observed that the Pdi is higher than 0.2 (not desired), the curves of the measurements are not perfectly superimposed, and a peak between 1000 and 10000 nm is observed, indicating the presence of a population of another size, which is not desired.

### Example 6

### Minimum Inhibitory Concentration (WC)

A lipid nanoparticle formulation loaded with amphotericin B (N439) was manufactured as described in Example 4 and it was used to determine the MIC (based on CLSI M27-A2 method). Susceptibility tests were performed using Candida albicans (American Type Culture Collection, USA; ATCC 10231) in order to compare the antifungal activity of the inventive nanoparticles substantially described in U.S. patent application Ser. No. 15/760,170 with the commercial liposomal formulation AMBISOME.

For this test, RPMI 1640 (Sigma-Aldrich, St Louis, Mo., USA; with glutamine, without bicarbonate, and with phenol red as a pH indicator), with glucose 0.2% and MOPS [3-(N,morpholino) propanesulfonic acid] at final concentration 0.165 mol/L, pH 7.0 culture medium was used. Also, the test was performed using sterile, 12×75 mm tubes and a growth control tube containing RPMI 1640 medium without any antifungal agents for the organism tested. A tube containing RPMI 1640 medium supplemented with antifungal agents without yeast was used as a turbidity control of the formulation.

### Example 7

### Capillary Electrophoresis

### Control of Protein (ApoE3) Binding to Nanoparticle

In order to control the protein (ApoE3) binding to nanoparticles a method of Capillary electrophoresis (CE) was used for applying ionic surfactants under MECC conditions. The control was made before and after the freeze-drying of the product with the ApoE3 incubated for 40 minutes with the lipid nanoparticle.

CE experiments were conducted on a PA800 Plus (Beckman Coulter, Fullerton, Calif., USA), equipped with a diode array detector (DAD) and an ultraviolet (UV) detector. A fused-silica capillary of 50 μm i.d.×60 cm (50 cm to detector) was used in separation. CE experiments were performed at 23° C. under optimum voltage settings (25 kV) and UV data were acquired using DAD. Prior to each run, the capillary was sequentially rinsed at 20 psi with 0.1 M NaOH for 3 min and miming buffer for 3 min.

Samples were injected under pressure at 0.5 psi for 10 seconds. The running buffers for separation of the nanoparticles and protein were prepared with 16 mM boric acid and 40 mM SDS pH 7.0 (carried to pH with NaOH 0.1M) (the reagents were purchased from Sigma-Aldrich). The unbound ApoE3 was quantified by standard addition previous calibration curve of ApoE3rec standard (purchased from AMEGA Biotech, Argentina). The following represents the equation by standard addition:

$$CMi == \frac{SMi * CSf}{ST - ( \frac{VM}{Vf} ) * SMi}$$

CMi=start concentration of sample (ApoE unbinding)

SMi=start signal of sample (ApoE unbinding)

CSf=final concentration of standard

ST=total signal

VM=sample volume

Vf=final volume.

### Example 8

### Uptake of the Lipid Nanoparticle by Different Tissues

A comparative analysis of tissue uptake was performed using Balb/c mice and the lipid nanoparticles loaded with amphotericin B both with and without ApoE3 labeled with1,1′-dioctadecyl-3,3,3′,3′-tetramethylindotricarbocyanine iodide (DiR, Santa Cniz Biotechnology Inc, Dallas, Tex.)a lipophilic fluorescent stains for hydrophobic structures.

For the trial, 0.06 ml of each formulation was inoculated in lateral veins of the tail of mice weighing 20 to 30 g. The clinical signs of the animals were evaluated 30 minutes post inoculation and throughout the entire trial. The distribution of fluorescent substances was analyzed in the Pearl Trilogy—LICOR System post-inoculation. The sacrifice and necropsy of one animal per group was performed to obtain images separately and evaluate the arrival of the lipid nanoparticle with ApoE3 in liver, brain, lungs and kidneys at 24, 48, 72 and 96hs after the sample administration with a last measurement at 8 days.

Additionally it was used a control with a solution of DiR without lipid nanoparticles. A control with a solution of DiR without lipid nanoparticles was tested.

In the brain, at 24 hours after the inoculation higher uptake for the ApoE3 formulation than for the formulation without ApoE3. In the liver, greater uptake of the formulation was observed with ApoE3 at 48 hours, decreasing in later hours, equaling the formulation without ApoE3.In lungs is where the greatest difference in the uptake is seen. At 24 hours and signal intensity of 3.15 was determined for the lipid nanoparticle with ApoE3 of 3.15 against a signal of 1.67 for the nanoparticle without ApoE3.

### Example 9

### Toxicity of the Lipid Nanoparticle with Amphotericin B and ApoE3

The toxicity of the formulation was assessed by an in vitro comparative hemolytic assay with a formulation of Amphotericin B in sodium deoxycholate (similar to FUNGIZONE in human cells). Hemolytic power of the formulations was tested using as reference the method described by Reed K. W, Yalkowsky S. H., “Lysis of human red blood cells in the presence of various cosolvents,” J. Parent. Sci. Tech. 39:64-9 (1985).

In 15 ml falcon tubes, 0.9 ml of human blood and 0.1 ml of the corresponding sample solution were added: a) amphotericin B in sodium deoxycholate (similar to FUNGIZOME) 0.08 mg/ml; b) Inventive Nanoparticle formulation with AmB 0.08 mg/ml; c) Normal Salt Solution (NSS) as negative control (0% Hemolytic action) d) 20% sodium carbonate solution (Na₂CO₃) as positive control (100% Hemolytic action). All samples were prepared in triplicate.

The mixture obtained was diluted with 5 ml of NSS, homogenized and centrifuged at 1500 rpm for 5 minutes to decant the intact erythrocytes and finally, the released hemoglobin was analyzed by UV spectrophotometer at 540 nm, carefully taking 1 ml of the supernatant with a micropipette from the top of the tube. The dilution volume was calculated so that the absorbance of the positive control was approximately 0.25, and the same dilution was applied for all samples. The following formula was used to obtain a % of hemolytic activity:

$$\% Hemolytic activity = \frac{( {Abs}_{M} - {Abs}_{Nss} )}{( {Abs}_{Na2CO3} - {Abs}_{Nss} )} \times 100$$

where:

Abss=sample absorbance

Abs N_{SS}=normal saline solution absorbance

Abs Na₂CO₃=sodium carbonate solution absorbance.

The results obtained at equal concentrations of AmB (0.8 mg/ml) show that the lipid nanoparticle with AmB and ApoE3 caused only 0.45% hemolysis compared to 19% produced by the similar FUNGIZONE formulation.

### Example 10

### Lipid Nanoparticles with Magnetite for Diagnosis Organic Phase Preparation

In a 500 ml Schott flask in a thermostated bath at 40° C. with nitrogen bubbling, 130 g of tert-butanol, 1.15 g of egg yolk phospholipids (Egg PC 80, Lipoid GmbH. Germany), 1.75 g of castor oil USP, 0.12 g of USP cholesterol and 5 ml of a magnetite solution of 9 nm in diameter were added. The mixture was homogenized by orbital shaking for 10 minutes.

Aqueous phase preparation: in a 2L glass reactor in a thermostated bath at 40° C. with nitrogen bubbling and mechanical stifling with glass paddles 750 g of water (WFI), 0.4 g of Poloxamer 188 (Lutrol F68, BASF Germany) and 0.2 g of Sodium Taurodeoxycholate (New Zealand Pharmaceuticals LTD, New Zealand) were added.

Nanoparticle Manufacture: to obtain the lipid nanoparticles, the organic phase was injected into the reactor containing the aqueous phase (preheated to 40° C. and with mechanical agitation) at a rate of 1-1.5 ml/sec using a 4-hole nozzle. Once the mixture was obtained, it was left in agitation for 45 minutes. The mixture containing the nanoparticles was concentrated by distillation under reduced pressure to reach the desired lipid concentration (approximately 25 mg/ml of total lipids). It was brought to pH 7.4 by the addition of a phosphate buffer solution.

Recombinant ApoE3 binding to lipid nanoparticles with magnetite: a volume of a 2 mg/ml solution of recombinant human ApoE3 was added to the reactor containing the concentrated magnetite nanoparticle solution to obtain a final concentration of 0.20 mg/ml of recombinant ApoE3 in the nanoparticle solution. The mixture was incubated in an oven at 40±2° C. with orbital shaking for one hour. It was measured for the obtained particles an average size of 148 nm and a PDI of 0.148.

### REFERENCES

Each of the references listed below and referenced in the disclosure is also incorporated herein by reference in its entirety.

Wagner et al., “Uptake Mechanism of ApoE-Modified Nanoparticles on Brain Capillary Endothelial Cells as a Blood-Brain Barrier Model,” PLoS ONE 7(3): e32568 (Mar. 1, 2012).

Kreuter et al., “Apolipoprotein-Mediated Transport of nanoparticle-Bound Drugs Across the Blood-Brain Barrier,” Journal of Drug Targeting, 10(4): 317-325 (2002).

Dehouck, Bénédicte, et al. “A new function for the LDL receptor: transcytosis of LDL across the blood-brain barrier,” The Journal of Cell Biology 138.4: 877-889 (1997).

Versluis, A. Jenny, et al. “Stable incorporation of a lipophilic daunorubicin prodrug into apolipoprotein E-exposing liposomes induces uptake of prodrug via low-density lipoprotein receptor in vivo,” Journal of Pharmacology and Experimental Therapeutics 289.1: 1-7 (1999).

Liu, Chia-Chan, et al. “Apolipoprotein E and Alzheimer disease: risk, mechanisms and therapy,” Nature Reviews Neurology 9.2: 106-118 (2013).

Vila-Rodriguez, Fidel, and William G. Honer. “ApoE and cholesterol in schizophrenia and bipolar disorder: comparison of grey and white matter and relation with APOE genotype.” Journal of Psychiatry& Neuroscience: JPN 36.1: 47 (2011).

BETTS et al. Environmental Toxicology and Chemistry, Vol. 32, No. 4, pp. 889-893

Narvekar, Mayuri, et al. “Nanocarrier for poorly water-soluble anticancer drugs-barriers of translation and solutions,” AAPS Pharm. SciTech 15.4: 822-833 (2014).

Gad, Shayne Cox, ed. Pharmaceutical manufacturing handbook: production and processes. Vol. 5. John Wiley & Sons, 2008.

Lockman, P. R., et al. “Nanoparticle technology for drug delivery across the blood-brain barrier,” Drug Development and Industrial Pharmacy 28.1 (2002): 1-13.

Mims, Martha P., Maurizio R. Soma, and Joel D. Morrisett. “Effect of particle size and temperature on the conformation and physiological behavior of apolipoprotein E bound to model lipoprotein particles,” Biochemistry 29.28: 6639-47 (1990).

Hatters, Danny M., Clare A. Peters-Libeu, and Karl H. Weisgraber. “Apolipoprotein E structure: insights into function,” Trends in Biochemical Sciences 31.8: 445-454 (2006).

Peters-Libeu, Clare A., et al. “Model of biologically active apolipoprotein E bound to dipalmitoylphosphatidylcholine,” Journal of Biological Chemistry 281.2 (2006): 1073-1079.

Robitaille, N., et al. “Apolipoprotein E polymorphism in a French Canadian population of northeastern Quebec: allele frequencies and effects on blood lipid and lipoprotein levels,” Human Biology, pp. 357-70 (1996).

Valdez, Rodolfo, et al. “Apolipoprotein E polymorphism and insulin levels in a biethnic population,” Diabetes Care 18.7: 992-1000 (1995).

Haffner, Steven M., et al. “Apolipoprotein E polymorphism and LDL size in a biethnic population,” Arteriosclerosis, Thrombosis, and Vascular Biology 16.9: 1184-88 (1996).

Utermann, Gerd, “Apolipoprotein E polymorphism in health and disease,” American Heart Journal 113.2: 433-440 (1987).

Feng, Lan, et al. “Development and optimization of oil-filled lipid nanoparticles containing docetaxel conjugates designed to control the drug release rate in vitro and in vivo,” Int. J. Nanomedicine 6: 2545 (2011).

Lundberg, B. B., et al. “A lipophilic paclitaxel derivative incorporated in a lipid emulsion for parenteral administration,” Journal of Controlled Release 86.1: 93-100 (2003).

Rodrigues, Debora G. et al., “Improvement of paclitaxel therapeutic index by derivatization and association to a cholesterol-rich microemulsion: in vitro and in vivo studies,” Cancer Chemotherapy and Pharmacology 55.6: 565-76 (2005).

Domb, Abraham J., Joseph Kost, and David Wiseman, eds. Handbook of biodegradable polymers. Vol. 7. CRC Press, 1998.

Patel, Hitesh R., Rakesh P. Patel, and M. M. Patel. “Poloxamers: A pharmaceutical excipients with therapeutic behaviors,” International Journal of PharmaTech Research 1.2: 299-303 (2009).

Frim, David M., et al. “The surfactant poloxamer-188 protects against glutamate toxicity in the rat brain,” Neuroreport 15.1: 171-74 (2004).

Coors, Esther A., et al. “Polysorbate 80 in medical products and nonimmunologicanaphylactoid reactions,” Annals of Allergy, Asthma & Immunology 95.6: 593-99 (2005).

Chaturvedi, S. P., and Vimal Kumar. “Production techniques of lipid nanoparticles: a review,” RJPBCS 3.3: 525-541 (2012).

Lasa-Saracibar, Beatriz, et al. “Lipid nanoparticles for cancer therapy: state of the art and future prospects,” Expert opinion on drug delivery 9.10: 1245-1261 (2012).

Hu, Fu-Qiang, et al. “Preparation and characteristics of monostearin nanostnictured lipid carriers,” International Journal of Pharmaceutics 314.1: 83-89 (2006).

Dehouck, Benedicte, et al., “A new function for the LDL receptor: transcytosis of LDL across the blood-brain barrier,” Journal of Cell Biology 138.4: 877-89 (1997).

Abdus Samad, Y. Sultana and M. Aqil, “Liposomal Drug Delivery Systems: An Update Review,” Current Drug Delivery 4: 297-305 (2007).

What is claimed is:

1. A method for enhancing transport of a therapeutic agent to a target cell or tissue, comprising:
administering to a subject a lipid nanoparticle loaded with the therapeutic agent, the lipid nanoparticle comprising:
a lipid core comprised of a triglyceride component and a cholesterol ester component;
the therapeutic agent;
a phospholipid layer;
a surfactant coating layer surrounding the phospholipid layer and the lipid core; and
a human recombinant apolipoprotein (ApoE3) adsorbed to a surface of the nanoparticle without Polysorbate 80,
wherein:
the lipid nanoparticle has preferential uptake in brain, lung, kidney and liver tissues that overexpress LDL receptors.

2. The method according to claim 1, wherein a molar ratio of the therapeutic agent molecules per each recombinant ApoE3 molecule in the lipid nanoparticle is in a range of from 45-140.

3. The method according to claim 1, wherein the therapeutic agent is loaded in the lipid nanoparticle without conjugation.

4. The method according to claim 1, wherein:
the target cell or tissue is a cell or tissue that over-expresses LDL receptors; and
the therapeutic agent is a diagnostic magnetic resonance imaging contrast agent that accumulates at the target tissue due to the over-expression of LDL receptors.

5. A method for enhancing transport of a therapeutic agent across a blood-brain barrier to a target cell or tissue, comprising:
administering to a subject a lipid nanoparticle loaded with the therapeutic agent, the lipid nanoparticle comprising:
a lipid core comprised of a triglyceride component and a cholesterol ester component;
the therapeutic agent;
a phospholipid layer;
a surfactant coating layer surrounding the phospholipid layer and the lipid core; and
human recombinant apolipoprotein (ApoE3) adsorbed to a surface of the nanoparticle without Polysorbate 80,
wherein:
the therapeutic agent is transported to the target cell or tissue in a concentration that is at least 10 times greater than a concentration transported by the same lipid nanoparticle without human recombinant ApoE3 adsorbed thereto.

6. The method according to claim 5, wherein the target cell or tissue is a cell or tissue of the brain, and the therapeutic agent is a drug that does not reach the target cell or tissue in a therapeutic window when administered without the lipid nanoparticle.

7. The method according to claim 5, wherein the therapeutic agent is at least one diagnostic magnetic resonance imaging contrast agent that accumulates at the target brain tissue, and the method further comprises obtaining at least one magnetic resonance image of the target brain tissue.

8. The method according to claim 7, wherein the therapeutic agent is a Gadolinium-based magnetic resonance imaging contrast agent.

9. The method according to claim 7, wherein the therapeutic agent is a magnetite-based magnetic resonance imaging agent coated with oleic acid coating.

10. The method according to claim 5, wherein the therapeutic agent is a chemotherapeutic drug and the target cell or tissue is of brain cancer.

11. A method of treating a disease associated with brain tissue, comprising:
administering a therapeutically effective amount of a therapeutic agent to an individual having the disease, the therapeutic agent being loaded onto lipid nanoparticles comprising:
a lipid core comprised of a triglyceride component and a cholesterol ester component;
a phospholipid layer;
a surfactant coating surrounding the phospholipid and the lipid core; and
a human recombinant apolipoprotein (ApoE3) adsorbed to a surface of the nanoparticle without Polysorbate 80, wherein the apolipoprotein is human recombinant ApoE3,
wherein the therapeutic agent is transported in the lipid nanoparticle across the blood-brain barrier to the target brain tissue through transcytosis independent of LDL receptor binding.

12. The method according to claim 11, wherein the therapeutic agent is an antibiotic and the disease is an intracerebral infection of Candida albicans.

13. The method according to claim 12, wherein the antibiotic is Amphotericin B.

14. The method according to claim 12, wherein the therapeutic agent is Amphotericin B that has at least 40% less toxicity in human red blood cells than a conventional formulation of Amphotericin B having a similar Minimum Inhibitory Concentration.

15. The method according to claim 11, wherein the therapeutic agent is a diagnostic magnetic resonance imaging contrast agent selected from Gadolinium-, Magnetite-, and. Fluorophore-based contrast agents.

16. The method according to claim 11, wherein the therapeutic agent is a chemotherapeutic drug for treatment of brain cancers.

17. The method according to claim 11, wherein the lipid nanoparticles loaded with the therapeutic agent are administered in a pharmaceutical composition, the pharmaceutical composition comprising the lipid nanoparticles and a pharmaceutically acceptable excipient.

18. The method according to claim 17, wherein the administration is intravenous or intranasal.

19. A method for treating skin conditions associated with reduced collagen production, comprising:
topically applying a composition comprising a therapeutically effective amount of lipid nanoparticles to an affected area on a surface of the skin, the lipid nanoparticles comprising:
a lipid core comprised of a triglyceride component and a cholesterol ester component;
a phospholipid layer;
a surfactant coating layer surrounding the phospholipid layer and the lipid core;
a human recombinant apolipoprotein (ApoE3) bonded to a surface of the nanoparticle without Polysorbate 80; and
at least one therapeutic agent in the lipid core,
wherein the nanoparticles diffuse from the surface of the skin across the epidermis, resulting in the therapeutic agent being intracellularly released in the dermis by LDL receptor-mediated endocytosis and stimulating fibrobplast collagen production.

20. The method for treating skin conditions according to claim 19, wherein the composition is in a form of a cream or a gel.

21. The method for treating skin conditions according to claim 19, wherein the therapeutic agent is Retinoin.

22. The method for treating skin conditions according to claim 22, wherein the therapeutic agent is Ingenol.
